SEQUENCE LISTING (16 sequences)

SEQ ID NO: 1 - protein, 114 aa, Homo sapiens
ASVTIRNAQTGRLLDSNYNGNVYTLPANGGNYQRWTGPGDGTVRNAQTGRCLDSNYDGAVYTLPCNGGSYQKWLFYSNGYIQNVETGRVLDSNYNGNVYTLPANGGNYQKWYTG

SEQ ID NO: 2 - protein, 113 aa, Artificial Sequence
  Actinohivin Variant Polypeptide
ASGTIRNAETGRLLDSNYDGAVYTLPANGGSYQRWTGPGDGTVRNAETGRLLDSNYDGAVYTLPANGGSYQKWTGPGDGTIQNAETGRLLDSNYDGAVYTLPANGGSYQKWTG

SEQ ID NO: 3 - protein, 113 aa, Artificial Sequence
  Actinohivin Variant Polypeptide
ASGTIRNAETGRCLDSNYDGAVYTLPCNGGSYQRWTGPGDGTVRNAETGRCLDSNYDGAVYTLPCNGGSYQKWTGPGDGTIQNAETGRCLDSNYDGAVYTLPCNGGSYQKWTG

SEQ ID NO: 4 - protein, 114 aa, Artificial Sequence
  Actinohivin Variant Polypeptide
ASVTIRNAETGRLLDSNYNGNVYTLPANGGNYQRWTGPGDGTVRNAETGRCLDSNYDGAVYTLPCNGGSYQKWLFYSNGYIQNVETGRVLDSNYNGNVYTLPANGGNYQKWYTG

SEQ ID NO: 5 - protein, 114 aa, Artificial Sequence
  Actinohivin Variant Polypeptide
ASVTIRNAETGRCLDSNYNGNVYTLPCNGGNYQRWTGPGDGTVRNAETGRCLDSNYDGAVYTLPCNGGSYQKWLFYSNGYIQNVETGRCLDSNYNGNVYTLPCNGGNYQKWYTG

SEQ ID NO: 6 - protein, 113 aa, Artificial Sequence
  Actinohivin Variant Polypeptide
ASGTIRNAETGRLLDSNYNGNVYTLPANGGNYQRWTGPGDGTVRNAETGRCLDSNYDGAVYTLPCNGGSYQKWTGPGDGTIQNAETGRVLDSNYNGNVYTLPANGGNYQKWTG

SEQ ID NO: 7 - protein, 113 aa, Artificial Sequence
  Actinohivin Variant Polypeptide
ASGTIRNAETGRCLDSNYDGNVYTLPCNGGSYQRWTGPGDGTVRNAETGRCLDSNYDGNVYTLPCNGGSYQKWTGPGDGTIQNAETGRCLDSNYDGNVYTLPCNGGSYQKWTG

SEQ ID NO: 8 - protein, 113 aa, Artificial Sequence
  Actinohivin Variant Polypeptide
ASGTIRNAQTGRCLDSNYNGNVYTLPCNGGNYQRWTGPGDGTVRNAQTGRCLDSNYDGAVYTLPCNGGSYQKWTGPGDGTIQNAETGRCLDSNYNGNVYTLPCNGGNYQKWTG

SEQ ID NO: 9 - protein, 113 aa, Artificial Sequence
  Actinohivin Variant Polypeptide
ASGTIRNAETGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTG

SEQ ID NO: 10 - protein, 113 aa, Artificial Sequence
  Actinohivin Variant Polypeptide
ASGTIRNAQTGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTG

SEQ ID NO: 11 - protein, 113 aa, Artificial Sequence
  Actinohivin Variant Polypeptide
ASGTIRNAETGRCLDSNYNGNVYTLPCNGGNYQRWTGPGDGTVRNAQTGRCLDSNYDGAVYTLPCNGGSYQKWTGPGDGTIQNAETGRCLDSNYNGNVYTLPCNGGNYQKWTG

SEQ ID NO: 12 - protein, 113 aa, Artificial Sequence
  Actinohivin Variant Polypeptide
ASGTIRNAQTGRLLDSNYNGNVYTLPANGGNYQRWTGPGDGTVRNAQTGRLLDSNYNGNVYTLPANGGNYQKWTGPGDGTIQNAQTGRVLDSNYNGNVYTLPANGGNYQKWTG

SEQ ID NO: 13 - protein, 113 aa, Artificial Sequence
  Actinohivin Variant Polypeptide
ASGTIRNAETGRLLDSNYNGNVYTLPANGGNYQRWTGPGDGTVRNAETGRLLDSNYNGNVYTLPANGGNYQKWTGPGDGTIQNAETGRVLDSNYNGNVYTLPANGGNYQKWTG

SEQ ID NO: 14 - protein, 113 aa, Artificial Sequence
  Actinohivin Variant Polypeptide
ASGTIRNAETGRCLDSNYNGNVYTLPCNGGNYQRWTGPGDGTVRNAETGRCLDSNYNGNVYTLPCNGGNYQKWTGPGDGTIQNAETGRCLDSNYNGNVYTLPCNGGNYQKWTG

SEQ ID NO: 15 - protein, 113 aa, Artificial Sequence
  Actinohivin Variant Polypeptide
ASGTIRNAETGRCLDSNYDGNVYTLPCNGGNYQRWTGPGDGTVRNAETGRCLDSNYDGNVYTLPCNGGNYQKWTGPGDGTIQNAETGRCLDSNYDGNVYTLPCNGGNYQKWTG

SEQ ID NO: 16 - protein, 350 aa, Artificial Sequence
  Actinohivin Variant Polypeptide Fused to An Amino Acid Sequence Comprising the Fc Region of IgG
ASGTIRNAETGRCLDSNYNGNVYTLPCNGGNYQRWTGPGDGTVRNAETGRCLDSNYDGAVYTLPCNGGSYQKWTGPGDGTIQNAETGRCLDSNYNGNVYTLPCNGGNYQKWTGGGGSVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0092841 A1
(43) Pub. Date: Mar. 21, 2024

(54) ACTINOHIVIN VARIANT POLYPEPTIDES AND RELATED METHODS

(71) Applicant: University of Louisville Research Foundation, Inc., Louisville, KY (US)

(72) Inventor: Nobuyuki Matoba, Owensboro, KY (US)

(21) Appl. No.: 18/261,950

(22) PCT Filed: Jan. 19, 2022
(86) PCT No.: PCT/US2022/012853

§ 371 Date: Jul. 18, 2023

Related U.S. Application Data
(60) Provisional application No. 63/139,115, filed on Jan. 19, 2021.

(51) Int. Cl. C07K 14/36; A61P 35/00

(52) U.S. Cl. CPC C07K 14/36; A61P 35/00; A61K 38/00

(57) ABSTRACT

The treatment of growth factor receptor-mediated cancers, such as epidermal growth factor receptor- (EGFR-) and/or insulin-like growth factor 1 receptor- (IGF1R-) mediated cancers, e.g., lung cancer such as non-small cell lung cancer, which is sensitive to polypeptides specifically binds a high-mannose-type glycan epitope, is described. Methods for reducing activation of EGFR and/or IGF1R in cancer cells, inhibiting cancer cell migration, treatment of cancer and/or reduction of tumor growth in subjects (e.g., human patients) are provided.

### RELATED APPLICATION(S)

This application claims the benefit of U.S. Provisional Application No. 63/139,115, filed on Jan. 19, 2021. The entire teachings of the above application(s) are incorporated herein by reference.

### GOVERNMENT SUPPORT

This invention was made with government support under 1R21CA216447-01 from National Institutes of Health (NIH)/National Cancer Institute (NCI), P30-GM106396 from National Institutes of Health (NIH), and W81XWH-10-2-0082-CLIN2 from the Department of Defense (DOD). The government has certain rights in the invention.

### INCORPORATION BY REFERENCE OF MATERIAL IN ASCII TEXT FILE

This application incorporates by reference the Sequence Listing contained in the following ASCII text file being submitted concurrently herewith:
a) File name: 56001008001PCT_Sequence_Listing_ST25.txt; created Dec. 6, 2021, 19000 Bytes in size.

### BACKGROUND

It has become evident that changes in protein glycosylation patterns are associated with various disease conditions, including viral infections and cancer.^{1, 2} One such change observed in several cancer types is a significant increase in the proportion of high-mannose-type glycans, which constitute a type of asparagine-linked glycan (N-glycan) containing 5-9 terminal mannose residues.^{3, 4} In normal cells, these glycoforms appear in the endoplasmic reticulum (ER) but are subsequently processed into complex-type glycans by a series of mannosidases and glycosyltransferases in the Golgi apparatus as nascent glycoproteins passage through the secretory pathway. Thus, high-mannose glycans are considered to be “immature” N-glycans that are generally confined in the ER under normal conditions.¹ However, recent studies based on quantitative mass spectrometry analyses of cancer tissue have demonstrated that this may not always be the case. For example, high-mannose glycans were elevated in serum samples from breast cancer patients, which correlated with cancer progression.⁵ Analysis of large cohorts of paired breast cancerous and adjacent non-tumor tissues found a high-mannose glycan (Man8) along with a triantennary glycan to be dramatically increased in the membrane fraction of tumors.⁶ Increased abundance of high-mannose glycans has also been observed in colorectal tumor tissues⁷⁻⁹, hepatocellular carcinoma^{10, 11}, metastatic cholangiocarcinoma², lung adenocarcinoma¹³, pancreatic cancer¹⁴, ovarian cancer^{15,16} prostate cancer¹⁷ and skin basal cell carcinoma and squamous cell carcinoma¹⁸. Collectively, the aberrant increase of high-mannose glycans on malignant cells may provide a unique biomarker for drug development. Nevertheless, there are few agents that can distinguish tumor-associated high-mannose glycans from other glycoforms present on a normal cell's surface, and thus their druggability remains unclear.

### SUMMARY

There is a critical need for identifying and characterizing new therapeutics that target cancer glycobiomarkers.

The subject matter disclosed herein is based, in part, on the discovery that “lectibody” AvFc is selective for oligomannose glycans overrepresented on the surface of cancer cells. Furthermore, AvFc is found to inhibit cancer-associated targets such as epidermal growth factor receptor (EGFR) and insulin-like growth factor 1 receptor (IGF1R), and mediate anti-cancer activities including antibody-dependent cell-mediated cytotoxicity (ADCC).

In one aspect, the disclosure provides a method of reducing activation of a growth factor receptor in a cancer cell, comprising contacting the cancer cell with a polypeptide that specifically binds a high-mannose-type glycan epitope.

In some embodiments, the growth factor receptor is a cancer-associated growth factor receptor. In particular embodiments, the growth factor receptor comprises an EGFR, an IGF1R, or a combination thereof.

In another aspect, the disclosure provides a method of inducing ADCC in a cancer cell, comprising contacting the cancer cell with a polypeptide that specifically binds a high-mannose-type glycan epitope.

In another aspect, the disclosure provides a method of inhibiting migration of a cancer cell, comprising contacting the cancer cell with a polypeptide that specifically binds a high-mannose-type glycan epitope.

In some embodiments, the cancer cell is a cell of a human patient.

In certain embodiments, the cancer cell is a non-small cell lung cancer (NSCLC) cell.

In another aspect, the disclosure provides a method of treating cancer in a subject in need thereof, comprising administering to the subject an effective amount of a polypeptide that specifically binds a high-mannose-type glycan epitope.

In another aspect, the disclosure provides a method of treating cancer in a subject in need thereof, comprising:
a) providing a biological sample from the subject;
b) determining presence or absence of an abnormal accumulation of a high-mannose glycan epitope in the biological sample; and
c) administering or providing for administration a therapeutically effective amount of a polypeptide that specifically binds the high-mannose-type glycan epitope to the subject if the abnormal accumulation is determined to be present in the biological sample.

In another aspect, the disclosure provides a method of treating cancer in a subject in need thereof, comprising administering to the subject an effective amount of a polypeptide that specifically binds a high-mannose-type glycan epitope, wherein the cancer is characterized by an abnormal cell-surface accumulation of high-mannose glycans.

In another aspect, the disclosure provides a method of treating cancer in a subject in need thereof, comprising administering to the subject an effective amount of a polypeptide that specifically binds a high-mannose-type glycan epitope, wherein the cancer is mediated by inappropriate activation of a growth factor receptor.

In another aspect, the disclosure provides a method of treating non-small cell lung cancer (NSCLC) in a subject in need thereof, comprising administering to the subject an effective amount of a polypeptide comprising an amino acid sequence set forth in SEQ ID NO:16.

In some embodiments, the growth factor receptor comprises an EGFR, an IGF1R, or a combination thereof.

In certain embodiments, the cancer is a lung cancer.

In particular embodiments, the cancer is resistant to treatment with an antibody that specifically binds a growth factor receptor.

In some embodiments, the polypeptide comprises an amino acid sequence that is at least about 90% identical to at least one sequence set forth in SEQ ID NOs:1-13.

In particular embodiments, the polypeptide comprises an amino acid sequence set forth in SEQ ID NO:9.

In certain embodiments, the polypeptide further comprises an Fc.

In particular embodiments, the polypeptide comprises an amino acid sequence set forth in SEQ ID NO:16.

In another aspect, the disclosure provides a method of reducing activation of a growth factor receptor in a cancer cell, comprising contacting the cancer cell with a polypeptide that specifically binds a highly glycosylated protein.

In another aspect, the disclosure provides a method of inducing ADCC in a cancer cell, comprising contacting the cancer cell with a polypeptide that specifically binds a highly glycosylated protein.

In another aspect, the disclosure provides a method of inhibiting migration of a cancer cell, comprising contacting the cancer cell with a polypeptide that specifically binds a highly glycosylated protein.

In another aspect, the disclosure provides a polypeptide for use in treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of a high-mannose glycan epitope in the biological sample; and
c) administering or providing for administration a therapeutically effective amount of the polypeptide to the subject if the abnormal accumulation is determined to be present in the biological sample.

In another aspect, the disclosure provides use of a polypeptide in manufacture of a medicament for treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of a high-mannose glycan epitope in the biological sample; and
c) administering or providing for administration a therapeutically effective amount of the polypeptide to the subject if the abnormal accumulation is determined to be present in the biological sample.

In another aspect, the disclosure provides a polypeptide for use in treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides a polypeptide for use in treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of the high-mannose glycan epitope in the biological sample; and
c) administering or providing for administration a therapeutically effective amount of the polypeptide that specifically binds the high-mannose-type glycan epitope to the subject if the abnormal accumulation is determined to be present in the biological sample.

In another aspect, the disclosure provides use of a polypeptide in manufacture of a medicament for treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of the high-mannose glycan epitope in the biological sample; and
c) administering or providing for administration a therapeutically effective amount of the polypeptide that specifically binds the high-mannose-type glycan epitope to the subject if the abnormal accumulation is determined to be present in the biological sample.

In another aspect, the disclosure provides use of a polypeptide in treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D show that AvFc recognizes high-mannose glycans on cancer cell lines. FIG. 1A Bindings of AvFc to various cancer cell lines, normal human peripheral blood mononucleous cells (PBMCs), and non-tumorigenic cell lines were evaluated by flow cytometry with 0.1, 1, or 10 mg/mL of drug. The percentages of FITC positive cells are shown as a heatmap, with most cell lines becoming saturated, at 10 mg/mL. FIG. 1B Immunofluorescence was used to visualize the binding of 1 mg/mL of AvFc to the non-tumorigenic lung epithelial cell line BEAS-2B and to A549 cells with or without endoglycosidase H (Endo H) treatments. AvFc does not show any binding to BEAS-2B or to endo H-treated A549 cells. FIGS. 1C-1D Flow cytometry of BEAS-2B and A549 after staining with either AvFc or Con A shows that Con A can weakly bind to both BEAS-2B and A549 cells, and that endo H digestion of cells abrogates binding by both lectins.

FIG. 2 shows inhibition of AvFc binding to cancer cells by HIV-1 gp120 and yeast mannan. A549 lung cancer cells were incubated with AvFc (1 μg/ml) and various concentrations of HIV-1 envelope glycoprotein gp120 and yeast mannan for 30 minutes at 4° C. Cells were washed and stained with 10 μg/ml of goat anti-human IgG FITC for 30 minutes at 4° C. Cells were then washed and analyzed for binding on a FACS Canto II (BD Biosciences) using FACSDiva (BD Biosciences).

FIGS. 3A-3G depict identification of putative cancer-cell-surface binding partners of AvFc. Potential binding partners were isolated using co-immunoprecipitation and identified using mass spectrometry. FIG. 3A Silver staining of AvFc and AvFc^{lec-} fractions obtained after co-immunoprecipitation. In addition to the band corresponding to AvFc itself (≈77 kDa), other species at higher and lower molecular weights are present suggesting that AvFc successfully pulled down potential binding partners. FIGS. 3B-D Co-immunoprecipitation was used to confirm the interaction between AvFc and EGFR/IGF1R isolated from A549 and H460 cells. Pull down with AvFc and then blotting with anti-EGFR or anti-IGFR antibodies revealed that AvFc, but not AvFc^{lec-}, interacts with EGFR and IGF1R derived from both cell lines. Note that a longer exposure time for IGF1R in A549, which was necessary due to low expression of the receptor, resulted in the detection of AvFc and AvFc^{lec-} in the blot due to cross-reactivity of the detection antibody. When the exposure time was extended in H460 IP, AvFc and AvFc^{lec-} bands appeared (right panel). FIG. 3E Potential binding partners isolated using co-immunoprecipitation and identified using mass spectrometry. FIG. 3F AvFc and Cetuximab (CTX) binding to monomeric and dimeric EGFRs. EGFR monomer and dimer were purified without and with 20 ng/ml of EGF treatments for 10 minutes, respectively, in A549 cells. To maintain a dimer form, EGFR dimer was cross-linked by BS³ linkers (Thermo Fisher Scientific, 21580) and filtered by 200 kDa cut-off filter (Advantec, USY-20). Vesicles composed of 1-palmitoyl-2-oleoyl-glycero-3-phosphocholine (POPC) and 1,2-dioleoyl-sn-glycero-3-phospho-L-serine (DOPS) (90:10 in molar ratio). Uniform sized (100 nm) vesicles were prepared by the vesicle extrusion method. EGFR (monomer) or EGFR (dimer) were inserted into vesicles (protein:lipids=2000:1 in molar ratio) in 0.7% of CHAPS added PBS. After dialysis in 2 μL of PBS, overnight at 4° C., varying concentrations of EGFR proteo-liposome was applied to an ELISA plate coated with 2 μg/ml of an anti-EGFR antibody (rabbit; Cell Signaling Technology, D38B1). The plate-captured EGFR proteo-liposomes were detected with 13.7 nM of AvFc or CTX and an anti-human IgG1-HRP conjugate (SouthernBiotech, 9054-05). FIG. 3G Expression levels of EGFR and IGF1R in lung cancer cell lines. EGFR and IGF1R expression were determined by immunoblot analysis using EGFR antibody and IGF1R antibody. Actin1 was included as a control.

FIGS. 4A-4G show that AvFc blocks EGFR and IGF1R signaling. The phosphorylation status of EGFR and IGF1R on A549 cells following treatment with their respective ligands was detected by anti-pEGFR(Y1068) and anti-IGF1R(Y1135/1136) antibodies. FIG. 4A A representative immunoblot shows that the treatment of A549 cells with 30 nM of AvFc and CTX, but not AvFc^{lec-}, prior to the addition of 2 ng/ml of EGF resulted in diminished EGFR activation. FIG. 4B Quantification of immunoblot in panel A. FIG. 4C A representative immunoblot shows that only treatment of A549 cells with 30 nM of AvFc, not CTX or AvFc^{lec-}, prior to the addition of 2 ng/ml of IGF1 results in decreased activation of IGF1R. FIG. 4D Quantification of immunoblot in panel C. FIG. 4E After incubation of A549 cells with AvFc, CTX or AvFc^{lec-} and subsequent stimulation with a mixture of EGF and IGF1, AKT and MAPK1 phosphorylation was only decreased with AvFc. FIG. 4F Quantification of pAKT/AKT in panel E. FIG. 4G Quantification of pMAPK1/MAPK1 in panel E. All relative band intensities were measured by ImageJ. Bars represent mean±SEM (N=3). Group means were compared with the two-tailed, unpaired Student t-test for FIGS. 4B and 4D, or one-way ANOVA with Bonferroni's posttests for FIGS. 4F and 4G (* p<0.05, ** p<0.01, *** p<0.001).

FIGS. 5A-5B show that AvFc inhibits A549 and H460 cell migration. Migration of A549 cells (FIG. 5A) or H460 cells (FIG. 5B) was measured in transwells with 8 μm pores after treatment of cells with 30 nM of AvFc, CTX, or AvFc^{lec-} and stimulation with EGF or IGF1. Error bars represent mean±SEM from three replicates. Groups were analyzed by two-way ANOVA followed by Bonferroni's Multiple Comparison Test (** p<0.01, *** p<0.001).

FIGS. 6A-6C show In vitro Fc-mediated anticancer activity of AvFc. FIGS. 6A-6B FcγRIIIa activation by AvFc, AvFc^{lec-} and CTX against A549 (FIG. 6A) or H460 (FIG. 6B) cells in a reporter cell-based assay. Representative data from at least two independent experiments are shown. Columns and bars represent mean±SEM (n=3). Groups were compared with two-way ANOVA followed by Tukey multiple comparison test (**p<0.01, ***p<0.001, ****p<0.0001). FIG. 6C is directed to a human PBMC-based ADCC assay using A549 lung cancer cells. Cells were pre-incubated with serial dilutions of AvFc or CTX for 30 min in a 37° C./5% CO₂ incubator. PBMCs were added to initiate the ADCC effects at an optimized effector/target ratio (50:1 for AvFc, 25:1 for CTX). After incubation in a 37° C./5% CO₂ incubator for 6 h, cell supernatants were collected for measuring released lactose dehydrogenase to calculate % target cell lysis. The experiment was done in triplicates, with mean±SEM shown for each data point.

FIGS. 7A-7E show In vivo anticancer activity of AvFc in the A549 subcutaneous xenograft challenge model (FIG. 7A) and the H460 subcutaneous xenograft challenge model (FIG. 7B) in SCID mice. Four days post challenge, mice were treated i.p. with AvFc or CTX at 25 mg/kg, or a vehicle control every other days (Q2D) for a total of 6 doses, as indicated by arrows. Animals were monitored until day 26 for A549 and day 16 for H460 models. Tumor volumes were compared with two-way ANOVA followed by Tukey multiple comparison tests (* p<0.05, ** p<0.01, *** p<0.001 between vehicle and AvFc; #p<0.05, ##p<0.01; ###p<0.001 between vehicle and CTX). FIGS. 7C-7D The A549-GFP human lung cancer metastasis model in SCID mice. SCID mice were i.v. challenged with A549-GFP cells on day 0, followed by every-other-day dosing with an i.p. injection of AvFc at 25 mg/kg (n=10) or a vehicle control (n=9), as indicated in the diagram. On day 35, the lungs were removed and GFP signal intensity of the lung from each mouse was quantified. Columns and bars represent mean±SEM, with dots representing individual mice (FIG. 7C). GFP images of the lungs (four right and single left lobes) from all animals in vehicle and AvFc groups are shown in FIG. 7D. Bar=5 cm. Statistical difference between groups was analyzed by the Mann-Whitney test. FIG. 7E Efficacy of AvFc in the B16F10 melanoma metastasis model. Briefly, animals were injected i.v. with 250,000 B16F10 cells on day 0. Treatment with 25 mg/kg (i.p.) of AvFc or AvFc^{lec-} (a non-sugar-binding mutant of AvFc) began on day 0 and continued every other day for a total of 6 doses. Animals were euthanized on day 21 and lung nodules were counted. While AvFc offered protection against tumor formation, the non-sugar-binding mutant AvFc^{lec-} failed to offer protection indicating that binding to high-mannose glycans is necessary for AvFc's anti-cancer activity.

FIGS. 8A-8E show IHC analysis of AvFc binding to primary human lung (NSCLC) tissue and EGFR. FIGS. 8A-8B IHC staining with AvFc or a biotinylated anti-human IgG secondary antibody only. Representative stains from Patient 117 lung tissues are shown, with hematoxylin as a counter stain. Bar=200 μm (FIG. 8A) or 100 μm (FIG. 8B). FIG. 8C Quantification of AvFc staining for lung tissues from all 10 patients tested using ImageJ. The number of positively stained cells between tumor and matched adjacent tissue was compared using the non-parametric Wilcoxon matched-pairs signed rank test (**p<0.01). FIG. 8D Representative immunoblot analysis of EGFR isolated from NSCLC tumor or matched adjacent tissue samples from 5 patients. EGFR was isolated by anti-EGFR IgG1 with Protein A bead precipitation and detected with AvFc^{lec-}, AvFc or CTX. CBB: Coomassie Brilliant Blue staining. FIG. 8E Quantification of EGFR immunoblot using a densitometory analysis. Relative binding intensities (tumor:adjacent) are shown for AvFc and CTX. Columns and bars represent mean SEM (n=5), and data were compared using an unpaired t-test (*p<0.05).

FIG. 9 shows the expression of EGFR and IGF1R in primary human lung tissues. IHC staining with anti-EGFR or anti-IGF1R. Representative stains from Patient 117 lung tissues are shown, with hematoxylin as a counter stain.

FIG. 10 shows impacts of mouse sera on AvFc's in vitro ADCC activity against A549 cells. The assay was performed as described in Example 1, except for the addition of serum from immunized (ADA titers 104-105) or non-immunized animals. In general, the addition of serum caused a slight increase in signal, with the serum containing the ADAs increasing it the most.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The disclosure is not limited by the illustrated ordering of acts or events, as some acts may occur in different orders and/or concurrently with other acts or events. Furthermore, not all illustrated acts, steps or events are required to implement a methodology in accordance with the disclosure. Many of the techniques and procedures described, or referenced herein, are well understood and commonly employed using conventional methodology by those skilled in the art.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

A description of example embodiments follows.

### Methods of the Disclosure

In one aspect, the disclosure provides a method of reducing activation of a growth factor receptor in a cancer cell, comprising contacting the cancer cell with a polypeptide that specifically binds a high-mannose-type glycan epitope.

In another aspect, the disclosure provides a method of reducing activation of a growth factor receptor in a cancer cell, comprising contacting the cancer cell with a polypeptide that specifically binds a highly glycosylated protein.

In certain embodiments, the growth factor receptor comprises at least one cancer-associated growth factor receptor, for example, 2, 3, 4, 5 or more cancer-associated growth factor receptors.

In some embodiments, the growth factor receptor comprises at least one tumor-associated growth factor receptor, for example, 2, 3, 4, 5 or more tumor-associated growth factor receptors.

In some embodiments, the growth factor receptor (e.g., cancer-associated growth factor receptor) comprises an epidermal growth factor receptor (EGFR). In certain embodiments, the growth factor receptor comprises an insulin-like growth factor 1 receptor (IGF1R). In particular embodiments, the growth factor receptor comprises an EGFR and an IGF1R. In some embodiments, the growth factor receptor comprises an ErbB family receptor (e.g., EGFR, ErbB2, ErbB3 and/or ErbB4), a fibroblast growth factor receptor (FGFR), IGF1R, IGF2R or a platelet-derived growth factor receptor (PDGFR), or a combination thereof.

In some embodiments, the EGFR is the human EGFR (also known as HER1 or ErbB1 (Ullrich et al., Nature 309:418-425, 1984) having the amino acid sequence shown in GenBank accession number NP_005219, NP 001333826, NP_001333827, NP_001333828, NP 001333829, NP_001333870, NP 005219, NP 958439, NP_958440 and NP_958441, as well as variants (e.g., naturally-occurring variants) thereof.

In some embodiments, the IGF1R is the human IGF1R (also known as CD221, IGFIR, IGFR or JTK13) having the amino acid sequence shown in GenBank accession number NP_000866.1, NP_001278787.1, EAX02222.1, XP 016877628.1, XP_016877625.1, XP_016877626.1, XP_016877627.1 or XP_011519818.1, as well as variants (e.g., naturally-occurring variants) thereof.

In certain embodiments, activation of the growth factor receptor (e.g., cancer-associated growth factor receptor such as EGFR and/or IGF1R) is reduced by at least about 10%, for example, by at least about: 15%, 20%, 25%, 30%, 35%, 40%, 45%, 50%, 55%, 60, 65%, 70%, 75%, 80%, 85% or 90%. In some embodiments, activation of the growth factor receptor is reduced by about 1-90%, for example, reduced by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In another aspect, the disclosure provides a method of inhibiting (e.g., slowing and/or reducing) cancer cell migration, comprising contacting the cancer cell with a polypeptide that specifically binds a high-mannose-type glycan epitope.

In another aspect, the disclosure provides a method of inhibiting (e.g., slowing and/or reducing) migration of a cancer cell, comprising contacting the cancer cell with a polypeptide that specifically binds a highly glycosylated protein.

In certain embodiments, migration of the cancer cell is inhibited (e.g., slowed and/or reduced) by at least about 10%, for example, by at least about: 15%, 20%, 25%, 30%, 35%, 40%, 45%, 50%, 55%, 60%, 65%, 70%, 75%, 80%, 85% or 90%. In some embodiments, migration of the cancer cell is inhibited by about 1-90%, for example, inhibited by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In particular embodiments, migration of the cancer cell is reduced by at least about 10%, for example, by at least about: 15%, 20%, 25%, 30%, 35%, 40%, 45%, 50%, 55%, 60%, 65%, 70%, 75%, 80%, 85% or 90%. In certain embodiments, migration of the cancer cell is reduced by about 1-90%, for example, reduced by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In another aspect, the disclosure provides a method of inducing ADCC in a cancer cell, comprising contacting the cancer cell with a polypeptide that specifically binds a high-mannose-type glycan epitope.

In another aspect, the disclosure provides a method of inducing ADCC in a cancer cell, comprising contacting the cancer cell with a polypeptide that specifically binds a highly glycosylated protein.

### Epitopes

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come into close proximity in a three-dimensional space through the folding of the protein molecule.

The term “high-mannose-type glycan” refers to asparagine-linked glycan (N-glycan) containing 5-9 terminal mannose residues attached to the chitobiose (GlcNAc₂) core. High-mannose glycans are formed and attached to newly synthesized nascent polypeptides containing asparagine-X-serine/threonine sequences, where X can be any amino acid except for proline, in the endoplasmic reticulum of eukaryotic cells. These glycans are then typically processed and matured into complex-type glycans containing fewer mannose residues as the nascent polypeptides undergo the secretory pathway through the Golgi apparatus. As a consequence, few high-mannose glycans remain attached to proteins that appear on the surface of healthy normal cells. However, unusually high-levels of high-mannose glycans are often found in cell-surface and secreted proteins produced by malignant cells.

Non-limiting examples of high-mannose-type glycans include: Man9GlcNAc2 (Man 9), Man8GlcNAc2 (Man8), Man7GlcNAc2 (Man 7), Man6GlcNAc2 (Man6) and Man5GlcNAc2 (Man5).

In some embodiments, the high-mannose-type glycan epitope is cancer-associated. In certain embodiments, the high-mannose-type glycan epitope is tumor-associated.

In particular embodiments, the high-mannose-type glycan epitope comprises one or more terminal α1,2-linked mannose residues.

In particular embodiments, the polypeptide specifically binds two or more high-mannose-type glycan epitopes, for example, 3, 4, 5 or more high-mannose-type glycan epitopes.

### Highly Glycosylated Proteins

In certain embodiments, the highly glycosylated protein comprises at least about 10 N-glycosylation sites, for example, at least about: 11, 12, 13, 14, 15, 16, 17 or 18 N-glycosylation sites. In particular embodiments, the highly glycosylated protein comprises about 13-16 N-glycosylation sites.

In some embodiments, the polypeptide specifically binds more than one highly glycosylated proteins, for example, 2, 3, 4, 5 or more highly glycosylated proteins.

In certain embodiments, the highly glycosylated protein comprises a highly glycosylated: agrin, cell-cycle control protein 50a, endothelial protein C receptor, epidermal growth factor receptor (EGFR), insulin-like growth factor 1 receptor (IGF1R), integrin alpha-1, integrin alpha-2, integrin alpha-3, integrin beta-5, laminin subunit alpha-5, laminin subunit beta-1, laminin subunit beta-2, low density lipoprotein receptor-related protein 1, neutral amino acid transporter b(0), protocadherin FAT1, solute carrier family12 member 7, or a combination thereof.

In some embodiments, the highly glycosylated protein comprises an EGFR. In certain embodiments, the highly glycosylated protein comprises an IGF1R. In particular embodiments, the highly glycosylated protein comprises an EGFR and an IGF1R.

### Polypeptide

The term “polypeptide” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L- and/or D-amino acid, for example, common α-amino acids (e.g., alanine, glycine, valine), non-α-amino acids (e.g., b-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

The term “specifically binding” or “specifically binds” refers to preferential interaction, i.e., significantly higher binding affinity, between an antibody, or an antigen-binding fragment thereof, and its epitope relative to other antigens or amino acid sequences.

In certain embodiments, the polypeptide is highly selective to malignant cells over noncancerous or normal healthy cells. For example, flow cytometry analysis showed that AvFc's 50% effective binding concentrations (EC₅₀s) for A549 and H460 human lung cancer cell lines were 42 ng/mL and 30 ng/mL, respectively, whereas EC₅₀ was >10 μg/mL for BEAS-2B non-tumorigenic lung epithelial cell line. In particular embodiments, the EC₅₀ of a polypeptide of the disclosure to a malignant cell is about 10-60 ng/mL, for example, about: 10-55 ng/mL, 15-55 ng/mL, 15-50 ng/mL, 20-50 ng/mL, 20-45 ng/mL, 25-45 ng/mL, 25-42 ng/mL or 30-42 ng/mL. In some embodiments, the EC₅₀ of the polypeptide to a malignant cell is about 30-42 ng/mL.

In some embodiments, the polypeptide comprises the wildtype actinohivin amino acid sequence (SEQ ID NO:1) or a variant thereof.

In certain embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:1-13.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:1. In particular embodiments, the polypeptide comprises an amino acid sequence set forth in SEQ ID NO:1.

In some embodiments, the polypeptide comprises an amino acid sequence that is at least about 75% (e.g., at least about: 80, 85, 90, 91, 92, 93, 94, 95, 96, 97, 98 or 99%) identical to at least one sequence set forth in SEQ ID NOs:2-13.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:2. In particular embodiments, the polypeptide comprises an amino acid sequence set forth in SEQ ID NO:2.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:3. In particular embodiments, the polypeptide comprises an amino acid sequence set forth in SEQ ID NO:3.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:4. In particular embodiments, the polypeptide comprises an amino acid sequence set forth in SEQ ID NO:4.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:5. In particular embodiments, the polypeptide comprises an amino acid sequence set forth in SEQ ID NO:5.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:6. In particular embodiments, the polypeptide comprises an amino acid sequence set forth in SEQ ID NO:6.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:7. In particular embodiments, the polypeptide comprises an amino acid sequence set forth in SEQ ID NO:7.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:8. In particular embodiments, the polypeptide comprises an amino acid sequence set forth in SEQ ID NO:8.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:10. In particular embodiments, the polypeptide comprises an amino acid sequence set forth in SEQ ID NO: 10.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:11. In particular embodiments, the polypeptide comprises an amino acid sequence set forth in SEQ ID NO:11.

In certain embodiments, the polypeptide comprises an amino acid sequence that is at least about 75% (e.g., at least about: 80, 85, 90, 91, 92, 93, 94, 95, 96, 97, 98 or 99%) identical to SEQ ID NO:12. In particular embodiments, the polypeptide comprises an amino acid sequence set forth in SEQ ID NO:12.

In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:13. In particular embodiments, the polypeptide comprises an amino acid sequence set forth in SEQ ID NO: 13.

In particular embodiments, the polypeptide comprises an amino acid sequence that is at least about 75% (e.g., at least about: 80, 85, 90, 91, 92, 93, 94, 95, 96, 97, 98 or 99%) identical to SEQ ID NO:9. In some embodiments, the polypeptide comprises an amino acid sequence set forth in SEQ ID NO:9.

In some embodiments, the polypeptide further comprises a fragment crystallizable domain of an antibody (Fc), a fragment antigen-binding domain of an antibody (Fab) or a single chain variable fragment of an antibody (scFv).

In certain embodiments, the polypeptide further comprises an Fab.

In some embodiments, the polypeptide further comprises a scFv.

In some embodiments, the polypeptide further comprises an Fc. In certain embodiments, the polypeptide comprises the high-mannose glycan-binding (actinomycete-derived, oligomannose-binding) lectin Avaren and IgG1 Fc (fragment crystallizable region (Fc) of human immunoglobulin G1) (the “lectibody” AvFc). In some embodiments, the polypeptide comprises an amino acid sequence that is at least about 75% (e.g., at least about: 80, 85, 90, 91, 92, 93, 94, 95, 96, 97, 98 or 99%) identical to SEQ ID NO:16. In particular embodiments, the polypeptide comprises an amino acid sequence set forth in SEQ ID NO:16.

### Cancer Cells

In some embodiments, the cancer cell is an in vitro cell.

In certain embodiments, the cancer cell is an ex vivo cell.

In some embodiments, the cancer cell is a cell of a subject (e.g., a human patient).

In certain embodiments, the cancer cell is a mammalian cell, e.g., a cell from a dog, a cat, a mouse, a rat, a hamster, a guinea pig, a horse, a pig, a sheep, a cow, a chimpanzee, a macaque, a cynomolgus, or a human. In some embodiments, the cancer cell is a primate cell. In particular embodiments, the cancer cell is a human cell.

In some embodiments, the cancer cell is a cell of a bile duct cancer (e.g., metastatic cholangiocarcinoma), a blood cancer (e.g., melanoma), a breast cancer (e.g., breast carcinoma), a cervical cancer (e.g., cervical carcinoma), a colon cancer (e.g., colon adenocarcinoma), a colorectal cancer, a liver cancer (e.g., hepatocellular carcinoma), a lung cancer (e.g., lung adenocarcinoma such as large-cell lung carcinoma), an ovarian cancer, a pancreatic cancer, a prostate cancer, or a skin cancer (e.g., skin basal cell carcinoma or squamous cell carcinoma).

In certain embodiments, the cancer cell is a lung cancer cell. In particular embodiments, the lung cell is a non-small cell lung cancer (NSCLC) cell.

In some embodiments, the cancer cell is a solid tumor cell selected from breast, lung, prostate, colon, bladder, ovarian, renal, gastric, rectal, colorectal, testicular, head and neck, pancreatic, brain and skin cancer cells.

In certain embodiments, the cancer cell is a hematologic cancer cell selected from leukemia (e.g., acute leukemias, chronic leukemias), lymphoma (e.g., B-cell lymphoma, T-cell lymphoma) and multiple myeloma cells.

In certain embodiments, the cancer cell is characterized by an abnormal surface accumulation of high-mannose glycans.

In some embodiments, the cancer cell expresses a protein with an abnormal accumulation of high-mannose glycans on its cell surface. In certain embodiments, said protein comprises a growth factor receptor, a laminin, an integrin, a transporter or a combination thereof. In some embodiment embodiments, said protein comprises agrin, cell-cycle control protein 50a, endothelial protein C receptor, epidermal growth factor receptor (EGFR), insulin-like growth factor 1 receptor (IGF1R), integrin alpha-1, integrin alpha-2, integrin alpha-3, integrin beta-5, laminin subunit alpha-5, laminin subunit beta-1, laminin subunit beta-2, low density lipoprotein receptor-related protein 1, neutral amino acid transporter b(0), protocadherin FAT1, solute carrier family12 member 7, or a combination thereof.

In some embodiments, the protein with an abnormal accumulation of high-mannose glycans comprises an EGFR. In certain embodiments, the protein with an abnormal accumulation of high-mannose glycans comprises an IGF1R. In particular embodiments, the protein with an abnormal accumulation of high-mannose glycans comprises an EGFR and an IGF1R.

In some embodiments, the cancer cell is characterized by one or more tumor-associated glycobiomarkers. In certain embodiments, the cancer cell is characterized by two or more tumor-associated glycobiomarkers.

In some embodiments, the cancer cell is characterized by inappropriate activation of a growth factor receptor. In some embodiments, the cancer cell is characterized by inappropriate activation of an EGFR. In certain embodiments, the cancer cell is characterized by inappropriate activation of an IGF1R. In particular embodiments, the cancer cell is characterized by inappropriate activation of an EGFR and an IGF1R.

In some embodiments, the cancer cell is resistant to an antibody that specifically binds a growth factor receptor, for example, an anti-EGFR antibody such as cetuximab (CTX).

### Methods of Treating Cancer

In another aspect, the disclosure provides a method of treating cancer in a subject in need thereof, comprising administering to the subject an effective amount of a polypeptide that specifically binds a high-mannose-type glycan epitope.

In another aspect, the disclosure provides a method of treating cancer in a subject in need thereof, comprising:
a) providing a biological sample from the subject;
b) determining presence or absence of an abnormal accumulation of a high-mannose glycan epitope in the biological sample; and
c) administering or providing for administration a therapeutically effective amount of a polypeptide that specifically binds the high-mannose-type glycan epitope to the subject if the abnormal accumulation is determined to be present in the biological sample.

In another aspect, the disclosure provides a method of treating cancer in a subject in need thereof, comprising administering to the subject an effective amount of a polypeptide that specifically binds a high-mannose-type glycan epitope, wherein the cancer is characterized by an abnormal cell-surface accumulation of high-mannose glycans.

In another aspect, the disclosure provides a method of treating cancer in a subject in need thereof, comprising administering to the subject an effective amount of a polypeptide that specifically binds a high-mannose-type glycan epitope, wherein the cancer is mediated by inappropriate activation of a growth factor receptor.

In another aspect, the disclosure provides a method of treating non-small cell lung cancer (NSCLC) in a subject in need thereof, comprising administering to the subject an effective amount of a polypeptide comprising an amino acid sequence set forth in SEQ ID NO:16.

In another aspect, the disclosure provides a polypeptide for use in treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of a high-mannose glycan epitope in the biological sample; and
c) administering or providing for administration a therapeutically effective amount of the polypeptide to the subject if the abnormal accumulation is determined to be present in the biological sample.

In another aspect, the disclosure provides use of a polypeptide in manufacture of a medicament for treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of a high-mannose glycan epitope in the biological sample; and
c) administering or providing for administration a therapeutically effective amount of the polypeptide to the subject if the abnormal accumulation is determined to be present in the biological sample.

In another aspect, the disclosure provides a polypeptide for use in treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides a polypeptide for use in treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of the high-mannose glycan epitope in the biological sample; and
c) administering or providing for administration a therapeutically effective amount of the polypeptide that specifically binds the high-mannose-type glycan epitope to the subject if the abnormal accumulation is determined to be present in the biological sample.

In another aspect, the disclosure provides use of a polypeptide in manufacture of a medicament for treating cancer in a subject in need thereof, wherein the polypeptide specifically binds a high-mannose-type glycan epitope, and wherein the method comprises:
a) providing a biological sample from the subject;
b) determining presence or absence of an abnormal accumulation of the high-mannose glycan epitope in the biological sample; and
c) administering or providing for administration a therapeutically effective amount of the polypeptide that specifically binds the high-mannose-type glycan epitope to the subject if the abnormal accumulation is determined to be present in the biological sample.

In another aspect, the disclosure provides use of a polypeptide in treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is characterized by an abnormal cell-surface accumulation of high-mannose glycans, wherein the polypeptide specifically binds a high-mannose-type glycan epitope, and wherein the method comprises administering to the subject an effective amount of the polypeptide.

In another aspect, the disclosure provides use of a polypeptide in treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

In another aspect, the disclosure provides use of a polypeptide in manufacture of a medicament for treating cancer in a subject in need thereof, wherein the cancer is mediated by inappropriate activation of a growth factor receptor, wherein the polypeptide specifically binds a high-mannose-type glycan epitope, and wherein the method comprises administering to the subject an effective amount of the polypeptide.

### Compositions

In some embodiments, a polypeptide described herein is provided in a composition, for example in a pharmaceutical composition.

In some embodiments, the composition (e.g., pharmaceutical composition) further comprises one or more pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the composition (e.g., pharmaceutical composition) of the disclosure is formulated for a suitable administration schedule and route. Non-limiting examples of administration routes include oral, rectal, mucosal, intravenous, intramuscular, subcutaneous and topical, etc. In some embodiments, the composition (e.g., pharmaceutical composition) of the disclosure is stored in the form of an aqueous solution or a dried formulation (e.g., lyophilized).

In some embodiments, the composition is formulated to be administered by infusion (e.g., intravenous infusion) or injection (e.g., intramuscular, subcutaneous, intraperitoneal or intratumoral injection). In certain embodiments, the composition is formulated to be administered by intravenous infusion. In some embodiments, the composition is formulated to be administered by intramuscular injection. In particular embodiments, the composition is formulated to be administered by subcutaneous injection. In some embodiments, the composition is formulated to be administered by intraperitoneal injection. In certain embodiments, the composition is formulated to be administered by intratumoral injection.

In some embodiments, the composition is formulated to be administered with one or more additional therapeutic agents as a combination therapy. Non-limiting examples of the one or more additional therapeutic agents include a T cell expressing chimeric antigen receptor (CAR) (CAR-T cell), a natural killer cell expressing CAR (CAR-NK cell), a macrophage expressing CAR (CAR-M cell), a chemotherapeutic agent, an immune checkpoint inhibitor, a T-cell redirector, radiation therapy, surgery and a standard of care drug.

Pharmaceutical composition referring to a product that results from combining a polypeptide that specifically binds a high-mannose-type glycan epitope and the one or more additional therapeutic agents includes both fixed and non-fixed combinations.

“Fixed combination” refers to a single pharmaceutical composition comprising two or more compounds, for example, the polypeptide that specifically binds a high-mannose-type glycan epitope and the one or more additional therapeutic agents are administered simultaneously in the form of a single entity or dosage. In some embodiments, a pharmaceutical composition comprising the polypeptide that specifically binds a high-mannose-type glycan epitope and the one or more additional therapeutic agents are provided as a fixed combination.

“Non-fixed combination” refers to separate pharmaceutical compositions, wherein each comprises one or more compounds, for example, the polypeptide that specifically binds a high-mannose-type glycan epitope and the one or more additional therapeutic agents are administered as separate entities either simultaneously, concurrently or sequentially with no specific intervening time limits, wherein such administration provides effective levels of the two or more compounds in the body of the subject. In some embodiments, pharmaceutical composition comprising the polypeptide that specifically binds a high-mannose-type glycan epitope and the one or more additional therapeutic agents are provided as a non-fixed combination.

In some embodiments, the polypeptide (e.g., AvFc) is systemically administered to the subject at about 10-50 mg/kg, for example, at about: 10 mg/kg, 15 mg/kg, 20 mg/kg, 25 mg/kg, 30 mg/kg, 35 mg/kg, 40 mg/kg, 45 mg/kg or 50 mg/kg, or at about: 10-45 mg/kg, 15-45 mg/kg, 15-40 mg/kg, 20-40 mg/kg, 20-35 mg/kg, 25-35 mg/kg or 25-30 mg/kg. In certain embodiments, the polypeptide (e.g., AvFc) is systemically administered to the subject at about 10-50 mg/kg about every 2-7 days (for example, about every: 2, 3, 4, 5, 6 or 7 days,) for about 2-10 weeks (for example, for about: 2, 3, 4, 5, 6, 7, 8, 9 or 10 weeks, or for about: 2-9, 3-9, 3-8, 4-8, 4-7, 5-7 or 5-6 weeks). In particular embodiments, the polypeptide (e.g., AvFc) is systemically administered to the subject at about 25 mg/kg of every other day (Q2D) for 14 or 20 days (8 or 11 doses total, respectively). In some embodiments, the polypeptide (e.g., AvFc) is systemically administered to the subject at about 10-50 mg/kg of every 7 days (Q7D) for 1-2 months.

### Cancers

“Cancer” refers to an abnormal growth of cells which tend to proliferate in an uncontrolled way and, in some cases, to metastasize (spread) to other areas of a patient's body.

In some embodiments, the cancer is a bile duct cancer (e.g., metastatic cholangiocarcinoma), a blood cancer (e.g., melanoma), a breast cancer (e.g., breast carcinoma), a cervical cancer (e.g., cervical carcinoma), a colon cancer (e.g., colon adenocarcinoma), a colorectal cancer, a liver cancer (e.g., hepatocellular carcinoma), a lung cancer (e.g., lung adenocarcinoma such as large-cell lung carcinoma), an ovarian cancer, a pancreatic cancer, a prostate cancer, or a skin cancer (e.g., skin basal cell carcinoma or squamous cell carcinoma).

In certain embodiments, the cancer is a lung cancer. In particular embodiments, the lung cancer is non-small cell lung cancer (NSCLC).

In some embodiments, the cancer is a solid tumor, e.g., breast, lung, prostate, colon, bladder, ovary, kidney, stomach, colon, rectum, testes, head and/or neck, pancreas, brain, or skin cancer.

In other embodiments, the cancer is a hematologic cancer, for example, leukemia, lymphoma, or myeloma. Hematologic cancers that can be treated according to the methods described herein include leukemias (e.g., acute leukemias, chronic leukemias), lymphomas (e.g., B-cell lymphoma, T-cell lymphoma) and multiple myeloma.

In certain embodiments, the cancer is characterized by an abnormal cell-surface accumulation of high-mannose glycans. In some embodiments, cell surface high-mannose glycans on a cancer cell of the disclosure are about 2-10 times higher than on a normal cell.

In some embodiments, the cancer is characterized by cell-surface expression of a protein with an abnormal accumulation of high-mannose glycans. In certain embodiments, said protein comprises a growth factor receptor, a laminin, an integrin, a transporter or a combination thereof. In some embodiment embodiments, said protein comprises agrin, cell-cycle control protein 50a, endothelial protein C receptor, epidermal growth factor receptor (EGFR), insulin-like growth factor 1 receptor (IGF1R), integrin alpha-1, integrin alpha-2, integrin alpha-3, integrin beta-5, laminin subunit alpha-5, laminin subunit beta-1, laminin subunit beta-2, low density lipoprotein receptor-related protein 1, neutral amino acid transporter b(0), protocadherin FAT1, solute carrier family12 member 7, or a combination thereof. In particular embodiments, the protein with an abnormal accumulation of high-mannose glycans comprises EGFR, IGF1R or both. In some embodiments, the protein with an abnormal accumulation of high-mannose glycans comprises EGFR. In certain embodiments, the protein with an abnormal accumulation of high-mannose glycans comprises IGF1R.

In some embodiments, the cancer is characterized by one or more tumor-associated glycobiomarker. In certain embodiments, the cancer is characterized by two or more tumor-associated glycobiomarkers.

In some embodiments, the cancer is mediated by inappropriate activation of a growth factor receptor. In some embodiments, the growth factor receptor comprises EGFR. In certain embodiments, the growth factor receptor comprises IGF1R. In particular embodiments, the growth factor receptor comprises EGFR and IGF1R.

### Subjects

The term “subject” refers to an animal (e.g., a mammal). In some embodiments, the subject is a mammal. In certain embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate. In particular embodiments, the subject is a human.

The terms “subject in need thereof” refers to a mammalian subject, preferably human, diagnosed with or suspected of having a disease (e.g., cancer such as a lung cancer), whom will be or has been administered a polypeptide according to a method of the invention. “Subject in need thereof” includes those subjects already with the undesired physiological change or disease as well as those subjects prone to have the physiological change or disease.

Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the subject is an adult patient. In certain embodiments, the subject is a juvenile patient. In particular embodiments, the subject is a pediatric patient.

In some embodiments, the subject is 18-75 years of age. In certain embodiments, the subject is 40 years of age or older, e.g., at least: 45, 50, 55, 60, 65, 70, 75, 80, 85 or 90 years old.

In certain embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject is 12 years of age or older.

In certain embodiments, the subject is two years of age or older, for example, at least: 3, 4, 5, 6, 7, 8, 9, 10, 11, 12 years of age or older. In some embodiments, the subject is 4 years of age or older. In some embodiments, the subject is 5 years of age or older. In some embodiments, the subject is 6 years of age or older.

In some embodiments, the subject has been diagnosed with cancer (e.g., a lung cancer) for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years or 10 years.

In particular embodiments, the subject is newly diagnosed with a cancer (e.g., a lung cancer). “Newly diagnosed” refers to a subject who has been diagnosed with cancer (e.g., a lung cancer) but has not yet received treatment for the cancer.

In certain embodiments, the subject is treatment naïve.

In some embodiments, the subject has received one or more prior anti-cancer therapies. In certain embodiments, the one or more prior anti-cancer therapies comprises one or more chemotherapeutic agents, checkpoint inhibitors, targeted anti-cancer therapies or kinase inhibitors, or any combination thereof.

In particular embodiments, the subject is relapsed or resistant to treatment with one or more prior anti-cancer therapies. “Refractory” refers to a disease that does not respond to a treatment. A refractory disease can be resistant to a treatment before or at the beginning of the treatment, or a refractory disease can become resistant during a treatment. “Relapsed” refers to the return of a disease or the signs and symptoms of a disease after a period of improvement after prior treatment with a therapeutic.

In some embodiments, the subject is resistant to treatment with an antibody that specifically binds a growth factor receptor, for example, an anti-EGFR antibody such as cetuximab (CTX).

### Treating

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

In some embodiments, the therapeutically effective amount of the polypeptide is sufficient to:
a) reduce activation of a growth factor receptor;
b) inhibit cancer cell migration;
c) induce a cytotoxic effect; or
d) slow tumor growth, or
a combination of the foregoing.

In some embodiments, the therapeutically effective amount of the polypeptide is sufficient to reduce activation of a growth factor receptor (e.g., EGFR, IGF1R, or both). In certain embodiments, the growth factor receptor comprises a cancer-promoting growth factor receptor (e.g., a tumor-promoting growth factor receptor). In particular embodiments, the growth factor receptor comprises two or more cancer-promoting growth factor receptors.

In certain embodiments, the therapeutically effective amount of the polypeptide is sufficient to reduce activation of EGFR. In some embodiments, the therapeutically effective amount of the polypeptide is sufficient to reduce activation of IGF1R. In particular embodiments, the therapeutically effective amount of the polypeptide is sufficient to reduce activation of EGFR and IGF1R by their respective ligands.

In certain embodiments, activation of the growth factor receptor (e.g., EGFR, IGF1R, or both) is reduced by at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, activation of the growth factor receptor is reduced by about 1-90%, for example, reduced by about 1-90%, for example, reduced by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In some embodiments, the therapeutically effective amount of the polypeptide is sufficient to inhibit (e.g., slow or reduce) cancer cell migration.

In certain embodiments, the therapeutically effective amount of the polypeptide is sufficient to slow or inhibit cancer cell migration mediated by activation of EGFR. In some embodiments, the therapeutically effective amount of the polypeptide is sufficient to slow or inhibit cancer cell migration mediated by activation of IGF1R. In particular embodiments, the therapeutically effective amount of the polypeptide is sufficient to slow or inhibit cancer cell migration mediated by activation of EGFR and IGF1R.

In certain embodiments, migration of the cancer cell is reduced by at least about 10%, for example, by at least about: 15%, 20%, 25%, 30%, 35%, 40%, 45%, 50%, 55%, 60%, 65%, 70%, 75%, 80%, 85% or 90%. In certain embodiments, migration of the cancer cell is reduced by about 1-90%, for example, reduced by about: 1-85%, 5-85%, 5-80%, 10-80%, 10-75%, 15-75%, 15-70%, 20-70%, 20-65%, 25-65%, 25-60%, 30-60%, 30-55%, 35-55%, 35-50% or 40-50%.

In some embodiments, the therapeutically effective amount of the polypeptide is sufficient to induce a cytotoxic effect. In certain embodiments, the cytotoxic effect comprises one or more Fc-mediated cytotoxic effects (ADCC).

In some embodiments, the therapeutically effective amount of the polypeptide is sufficient to slow tumor growth.

### Diagnosis

In certain embodiments, methods of treatment further comprise determining if a biological sample of the subject in need is characterized with high-mannose-type glycan epitopes.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

In some embodiments, the method further comprises:
a) providing a biological sample from the subject; and
b) determining presence or absence of an abnormal accumulation of the high-mannose glycan epitope in the biological sample.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

In certain embodiments, the biological sample is a blood sample. In some embodiments, the biological sample is a tumor tissue biopsy.

In certain embodiments, the one or more proteins comprise a growth factor receptor, a laminin, an integrin, a transporter or a combination thereof. In some embodiment embodiments, said protein comprises agrin, cell-cycle control protein 50a, endothelial protein C receptor, epidermal growth factor receptor (EGFR), insulin-like growth factor 1 receptor (IGF1R), integrin alpha-1, integrin alpha-2, integrin alpha-3, integrin beta-5, laminin subunit alpha-5, laminin subunit beta-1, laminin subunit beta-2, low density lipoprotein receptor-related protein 1, neutral amino acid transporter b(O), protocadherin FAT1, solute carrier family12 member 7, or a combination thereof. In particular embodiments, the one or more proteins comprise EGFR, IGF1R or both. In some embodiments, the one or more proteins comprise EGFR. In certain embodiments, the one or more proteins comprise IGF1R.

### Embodiments

1. A method of reducing activation of epidermal growth factor receptor (EGFR) or insulin-like growth factor 1 receptor (IGF1R), or both in a cancer cell, the method comprises contacting the cancer cell with an effective amount of a polypeptide comprising an actinohivin variant.

2. The method of Item 1, wherein the polypeptide reduces activation of EGFR in the cancer cell.

3. The method of Item 1 or 2, wherein the polypeptide reduces activation of IGF1R in the cancer cell.

4. A method of inhibiting cancer cell migration, the method comprises contacting the cancer cell with an effective amount of a polypeptide comprising an actinohivin variant.

5. The method of any one of Items 1-4, wherein the cancer cell is a non-small cell lung cancer (NSCLC) cell.

6. The method of any one of Items 1-5, wherein the cancer cell is resistant to an anti-EGFR antibody.

7. A method of treating cancer in a subject in need thereof, comprising administering to the subject an effective amount of a polypeptide comprising an actinohivin variant, wherein the cancer is epidermal growth factor receptor- (EGFR-) mediated or insulin-like growth factor 1 receptor- (IGF1R-) mediated, or a combination thereof.

8. The method of Item 7, wherein the cancer is mediated by EGFR.

9. The method of Item 7 or 8, wherein the cancer is mediated by IGF1R.

10. The method of any one of Items 7-9, wherein the cancer is lung cancer.

11. The method of Item 10, wherein the lung cancer is non-small cell lung cancer (NSCLC).

12. The method of any one of Items 7-11, wherein the cancer is resistant to an anti-EGFR antibody.

13. The method of any one of Items 1-12, wherein the actinohivin variant comprises an amino acid sequence selected from the group consisting of SEQ ID NOs:2-13, and optionally, the actinohivin variant comprises an amino acid sequence set forth in SEQ ID NO:9.

14. The method of any one of Items 1-13, wherein the polypeptide further comprises a fragment crystallizable domain of an antibody (Fc), and optionally, the polypeptide comprises an amino acid sequence set forth in SEQ ID NO:16.

15. The method of any one of Items 1-13, wherein the polypeptide further comprises a fragment antigen-binding domain of an antibody (Fab) or a single chain variable fragment of an antibody (scFv).

### EXAMPLES

Aberrant protein glycosylation is a hallmark of cancer, but few drugs targeting cancer glycobiomarkers are currently available. The Inventor demonstrated that a “lectibody,” a translational fusion protein consisting of the high-mannose glycan-binding (actinomycete-derived, oligomannose-binding) lectin Avaren and IgG1 Fc (fragment crystallizable region (Fc) of human immunoglobulin G1) (the “lectibody” AvFc) selectively recognizes a range of cell lines derived from various cancers, including lung, breast, colon and blood cancers, at nanomolar concentrations. AvFc's binding to the non-small cell lung cancer (NSCLC) cell lines A549 and H460 was characterized in detail. Co-immunoprecipitation proteomics analysis revealed that epidermal growth factor receptor (EGFR) and insulin-like growth factor 1 receptor (IGF1R) are among the lectibody's common targets in these cells. AvFc blocked the activation (e.g., phosphorylation and downstream signaling) of EGFR and IGF1R by their respective ligands in A549 cells. Additionally, AvFc inhibited the migration of A549 and H460 cells upon stimulation with EGF and IGF1. Furthermore, AvFc induced potent Fc-mediated cytotoxic effects and significantly retarded A549 and H460 tumor growth in SCID mice. Immunohistochemistry analysis of primary lung tissues from NSCLC patients demonstrated that AvFc preferentially binds to tumors over adjacent non-tumor tissues. These results suggest that AvFc elicits anti-cancer activity through Fc-mediated effector functions along with the inhibition of tumor-promoting growth factor receptors. These findings provide evidence that increased abundance of high-mannose glycans in the glycocalyx of cancer cells can be a druggable target, and AvFc provides a new tool to probe and target this tumor-associated glycobiomarker.

There are few agents that can distinguish tumor-associated high-mannose glycans from other glycoforms present on a normal cell's surface, and their druggability remains unclear.

Previously, an antibody-like “lectibody” molecule comprised of the oligomannose-specific Avaren lectin and the fragment crystallizable region (Fc) of human IgG1, called Avaren-Fc (AvFc), was created.¹⁹ Avaren is an engineered variant of the actinomycete-derived antiviral lectin actinohivin,^{20,21} with amino acid substitutions to improve solubility and producibility. AvFc neutralized the infectivity of multiple HIV strains and hepatitis C viruses at nanomolar concentrations through high-affinity binding to high-mannose glycans clustered on their envelope glycoproteins.^{19,22} Additionally, the lectibody exhibited antibody-dependent cell-mediated virus inhibition against HIV-infected peripheral blood mononuclear cells (PBMCs) via its capacity to interact with activating Fcγ receptors such as FcγRI and FcγRIIIa. Preliminary safety studies in mice and rhesus macaques showed that systemic administration of AvFc did not induce any discernable toxicity.¹⁹ Furthermore, systemic administration of 25 mg/kg of AvFc every other day (Q2D) for 14 or 20 days (8 or 11 doses total, respectively) completely protected against HCV challenge without causing hepatotoxicity or any other significant adverse effects in a chimeric human liver mouse model.²² These results lend support for the use of AvFc in novel therapeutic strategies targeting high-mannose glycans that may loom on the cell surface in high densities under pathological conditions.

In light of growing evidence for the abberant overexpression of high-mannose glycans in neoplastic cells, it is hypothesized that AvFc could efficiently recognize these mannose-rich glycans on the surface of cancer cells and thereby exhibit antitumor activity. To address this hypothesis, Examples 2-7 investigated the lectibody's capacity to target cancer using human non-small cell lung cancer (NSCLC) cell lines, murine xenograft models of human NSCLC and primary human NSCLC tissue sections. The results provide support for a novel anti-cancer strategy targeting tumor-associated high-mannose glycans.

### Example 1. Materials and Methods

Human Lung Tissues

De-identified post-operative human lung cancer tissues and paired adjacent tissues were acquired from University of Louisville hospital (Louisville, KY). The pathological type of each tumor was determined to be NSCLC. Informed written consent was provided by all participants and the study protocol was approved by the Human Subjects Protection Program of University of Louisville (Study #18.1240). The distinction between tumor and adjacent tissue was made by the surgeon at the time of tissue removal, and tissues were immediately frozen in liquid nitrogen at the surgery and stored at −80° C.

Animal Housing and Care

Nine-week-old female PrkdC^{scid}/SzJ (SCID) mice (The Jackson Laboratory, Bar Harbor, ME) were housed in a temperature-controlled environment, with an alternating light/dark cycle of 12 hours and free access to standard diet and water. The investigators were not blinded for sample administration. All experimental procedures were approved by the University of Louisville's Institutional Animal Care and Use Committee.

Reagents

Antibodies specific to EGFR (D38B1), phospho-EGFR (Y1068), IGF1R (D23H3), phospho-IGF1R (Y1131), AKT, phospho-AKT (S473), MAPK1, and phospho-MAPK1 (ERK1/2) were purchased from Cell Signaling Technology (Danver, MA). EGF and IGF1 were purchased from Thermo Fisher Scientific (Waltham, MA). CTX was obtained from the University of Louisville Hospital pharmacy.

Cell Culture

All cell lines were obtained from American Type Culture Collection (ATCC, Manassas, VA) and authenticated by the supplier. Cells were grown according to ATCC's recommendations, regularly screened for mycoplasma using a commercial PCR-based kit (ATCC, Manassas, VA) and tested at low passage numbers, with quality ensured based on viability and morphologic inspection. In particular, A549 cells were grown in DMEM supplemented with 10% fetal bovine serum (FBS) and 1% penicillin/streptomycin and H460 cells were grown in RPMI 1640 supplemented with 10% FBS and 1% penicillin/streptomycin unless otherwise stated.

Production of AvFc

AvFc and AvFc^{lec-} were produced using a transient plant expression vector in Nicotiana benthamiana as described previously.¹⁹ Briefly, 4-week old plants were transformed with a magnICON® vector containing the gene for AvFc by agroinfiltration and incubated for one week. At that time, leaf tissue was homogenized in a NaPi buffer at a pH of 7.4 and clarified by centrifugation, followed by fast protein liquid chromatography on the ÅKTA pure system (GE Healthcare Life Sciences, Chicago, IL) using protein A as the first chromatography step and ceramic hydroxyapatite (CHT) as a cleanup step. Endotoxin was removed from the purified protein using the Triton X-114 phase separation method, followed by concentration of the protein using a 10 kDa MWCO centrifuge filter and sterilization with a 0.2 μm filter. Purity was assessed with SDS-PAGE, with AvFc appearing as a band at approximately 77 kDa under non-reducing conditions.

Flow Cytometry Analysis of AvFc Binding to Human Cells

Cancer-cell lines were harvested and incubated with various concentrations of AvFc (0.1, 1 and 10 μg/mL) in culture medium for 30 minutes on ice and washed 3 times with DPBS. Cells were then incubated with goat F(ab′)₂ anti-Human IgG Fc-FITC antibody (Abcam, Cambridge, MA) for 30 minutes in the dark on ice. After washing 3 additional times with DPBS, the cells were fixed with 1% formalin for 15 minutes on ice. Data were acquired on a FACSCalibur flow cytometer (BD BioSciences, San Jose, CA) by counting 10,000 events per sample and determining the percentage of FITC⁺ cells with FlowJo. The non-sugar-binding mutant AvFc^{lec-} was used as a negative control. The analyses were performed in triplicate.

Immunofluorescence

1,000 A549 cells or 10,000 BEAS-2B cells were seeded per chamber in Lab-Tek II chamber slides (Thermo Fisher Scientific, Waltham, MA) and incubated for 24 hours. After washing with PBS, cells were fixed in 4% formalin in PBS for 20 minutes at room temperature. After incubation with 0.2% Triton X-100 in PBS for 15 minutes at room temperature, Human Fc Block™ (BD, San Jose, CA) was added to cells and incubated for an additional 10 minutes. Cells were then blocked with 3% BSA-PBS for 30 minutes at room temperature and then incubated with 250 units of endoglycosidase H at 37° C. for 1 hour, according to the manufacturer's protocol (New England Biolabs, Ipswich, MA). Cells were then stained with 10 μg/ml of AvFc for 3 hours at room temperature and, after washing with PBS, stained with a 1:40 dilution of anti-human IgG-FITC (Sigma, Mendota Heights, MN) for 1 hour at room temperature. Cells were then mounted with coverslips using mounting medium for fluorescence with DAPI (VECTASHIELD®, Burlingame, CA). Slides were analyzed by fluorescent confocal microscopy (ZEISS LSM 880).

Co-Immunoprecipitation

1×10⁶ A549 or H460 cells were seeded in a 10 cm² plate (Corning, Tewksbury, MA) and incubated in growth medium for 24 hours. Cells were washed with PBS and cell lysates were prepared in T-PER buffer (Thermo Fisher Scientific, Waltham, MA) supplemented with a protease/phosphatase inhibitor (Thermo Fisher Scientific, Waltham, MA). After centrifugation at 13,000×g for 10 minutes at 4° C., supernatants were mixed with 4 μg of AvFc or AvFc-le. After incubation for 24 hours at 4° C., 10 μl of protein A beads (Santa Cruz, Dallas, TX) were added. After an additional incubation for 2 hours at 4° C., the mixture was washed with T-PER buffer and immune-blot analysis was performed.

EGFR Isolation

Tissue homogenates were prepared by silicon beads and Precellys® 24 homogenizer (Bertin, Rockville, MA) in T-PER buffer (Thermo Fisher Scientific, Waltham, MA) with protease inhibitor cocktail (Sigma, Mendota Heights, MN). Debris were removed by centrifugation at 13,000×g for 10 minutes at 4° C. Supernatants were incubated with 4 μg of Anti-EGFR IgG1 (D38B1) (Cell Signaling Technology, Danver, MA) and 20 μg of protein A beads (Santa Cruz, Dallas, TX) for 4 hours at 4° C. The mixture was washed with T-PER buffer.

Immunoblot Analysis

SDS-PAGE and membrane transfer cassettes were purchased from Thermo Fisher Scientific (Waltham, MA). Protein samples were run on 10% Bolt Bis-Tris Plus gels with NuPAGE MES SDS running buffer (Thermo Fisher Scientific, Waltham, MA). Transfer to PVDF membranes in NuPAGE transfer buffer was carried out at 10 V overnight at 4° C. Membranes were then incubated in 3% BSA in TBST for 2 hours and anti-EGFR, anti-IGF1R, anti-human Fc, or AvFc in TBST supplemented with 1% BSA were incubated over-night at 4° C. HRP tagged secondary antibodies (Anti-rabbit IgG, Santa Cruz, Dallas, TX; Anti-human IgG, SouthernBiotech, Birmingham, AL) were used for the protein detection and membrane images were taken using Amersham Imager 600 (GE Healthcare Life Sciences, Chicago, IL).

Proteomics Analysis

Potential cell-surface binding partners of AvFc were identified using co-immunoprecipitation followed by mass spectrometry. Co-immunoprecipitation in this instance was performed using the Pierce Co-immunoprecipitation Kit (Thermo Fisher Scientific, Waltham, MA) according to the manufacturer's instructions. Briefly, whole-cell lysates of A549 and H460 cells were pre-cleared with a control agarose resin to reduce non-specific interactions, then co-incubated with 100 μg of AvFc or AvFc^{lec-} which were covalently attached to an agarose resin for 2 hours at 4° C. Bound proteins were then eluted using a low pH buffer and neutralized with 1 M tris base for mass spec analysis.

Protein samples were digested with trypsin (1:50 ratio) in a filter-aided sample preparation approach following reduction and alkylation with 100 mM dithiotheritol and 50 mM iodoacetamide. The tryptic digests (0.5 mg) were separated using a Proxeon EASY n-LC (Thermo-Fisher Scientific) UHPLC system and Dionex (Sunnyvale, CA) 2 cm Acclaim PepMap 100 trap and a 15 cm Dionex Acclaim PepMap RSLC (C18, 2 μm, 100 Å) separating column. The eluate was introduced into an LTQ-Orbitrap ELITE (Thermo-Fisher Scientific) using a Nanospray Flex source and MS2 data collected in a data dependent fashion in a top-20 rapid CID method. All MS1 data were acquired using Fourier transform ion cyclotron resonance MS at 240,000 resolution and MS2 data using the linear ion trap. MSn data were searched using Proteome Discoverer 1.4 (Thermo Scientific) with Sequest HT (SageN) and Mascot, version 4.0 (Matrix Science) in a decoy database search strategy against UniProt Knowledgbase, Homo sapiens reference proteome. The searches were performed with a fragment ion mass tolerance of 1.0 Da and a parent ion tolerance of 50 ppm. The search data results files were imported into Scaffold, version 4.3.4 (Proteome Software Inc.) and filtered using a 2 ppm mass error filter, removal of decoy hits, to control for <1.0% false discovery rates with PeptideProphet and ProteinProphet (Institute for Systems Biology). Peptide and protein identifications were accepted at >95.0% probability by the PeptideProphet or ProteinProphet algorithm. A comparison of protein abundance among the sample sets was conducted in Scaffold using the intensity based absolute quantification (iBAQ) method. Results were further refined using Gene Ontology (GO) terms to extract the most abundant membrane receptors, transporters, and adhesion molecules bound by AvFc and not AvFc^{lec-}.

Transwell Migration Assay

1×10⁵ A549 or H460 cells in 200 μl of serum-free growth medium were seeded in the insert of a transwell plate with 8 μm pores (VWR International, Radnor, PA). These cells were coincubated with AvFc or CTX at 30 nM for 2 hours at 37° C. Afterwards, growth medium supplemented with 20% FBS was added to the outside well and EGF or IGF-1 was added to a final concentration of 2 ng/mL in the transwell insert. After 6 hours, migrated cell counts were determined by trypsinization and trypan blue staining (Thermo Fisher Scientific, Waltham, MA).

ADCC Reporter Assay

Antibody-dependent cell-mediated cytotoxicity (ADCC) was assessed by an ADCC Reporter Bioassay (Promega, Madison, WI) following the manufacturer's protocol. Each sample was tested in triplicate. Briefly, 3 NSCLC cell lines used as target cells were seeded in an opaque white 96-well flat-bottom culture plate (Corning, Tewksbury, MA) at 10,000⁴ cells/well and incubated at 37° C. with 5% CO₂. 24 hours later, various concentrations of AvFc, AvFc^{lec-}, or CTX were added to target cells along with the Jurkat NFAT-luc FcγRIIIa-expressing cell line (Jur-FcγRIIIa; Promega, Madison, WI) at a ratio of 15:1. FcγRIIIa signaling activates the NFAT transcription factor, inducing the expression of firefly luciferase through an NFAT responsive promoter. After co-culture for 24 hours, firefly luciferase activity was measured using the Britelite Plus Reporter Gene Assay System (Perkin Elmer, Waltham, MA) on a Synergy HT luminometer (BioTeck, Winooski, VT). Jur-FcγRIIIa cells co-cultured with the target cells in the absence of antibody provided no antibody control luciferase production levels, which were subtracted from the actual signals to yield antibody-specific activation, in relative light units (RLUs). Background was determined by taking the mean of the target-cell-only wells. Fold induction was calculated using the following equation:

Fold induction=(RLU_{induced}−RLU_{background})/(RLU_{noAbcontrol}−RLU_{background}).

ADCC Assay with Primary Human PBMC Effector Cells

Similar to the reporter assay, plated A549 target cells were pre-incubated with serial dilutions of AvFc or CTX for 30 min at 37° C. PBMCs were added to initiate the ADCC at ratio of 50:1 for AvFc and 25:1 for CTX. After incubation at 37° C. incubator for 6 h, cell supernatants were collected, and the released lactose dehydrogenase was measured and compared to a no-drug control to calculate % target cell lysis. Each sample was tested in triplicate.

Subcutaneous Lung Cancer Xenograft Challenge Model

1×10⁷ A549 cells or 5×10⁵ H460 were implanted into the hind-left flanks of 8-week-old female SCID mice. Mice were then randomly organized into three groups and treated with the vehicle (n=10), 25 mg/kg AvFc (n=10), and 25 mg/kg CTX (n=10). Vehicle treatment consisted of the AvFc formulation buffer (30 mM histidine pH 7.4, 100 mM sucrose, 100 mM NaCl). Treatments were administered i.p. on days 4, 6, 8, 10, 12, and 14 following the formation of palpable lesions. Body weights and tumor volumes were measured every other day after treatment. Animals were euthanized 26 days after A549 challenge and 16 days after H460 challenge.

A549 Lung Metastasis Model

A549 cells expressing GFP were grown to confluency in growth medium. After harvest, 2×10⁶ cells were injected i.v. into 9-week-old female SCID mice. Mice were then randomly organized into two groups and treated with the vehicle (n=10), and 25 mg/kg AvFc (n=10). Treatments were administered i.p. on days 0, 2, 4, 6, 8, and 10. Following treatment, mice were weighed every other day. Finally, the animals were euthanized on 28 days, with the lungs surgically removed and fixed in 10% formalin. To detect GFP signals, each lobe of lungs was separated and GFP signals were detected by Amersham Imager 600 (GE Healthcare Life Sciences, Chicago, IL).

Immunohistochemistry

Immunohistochemical staining was performed on cryo-sections of frozen tissues from lung patients undergoing surgery. Staining was performed with the VECTASTAIN® Elite® ABC HRP Kit (Peroxidase, Standard) (Vector Labs, Burlingame, CA). 8-μm tissue sections were placed on positively charged slides (VWR International, Radnor, PA) and air dried. Then, sections were incubated for 10 minutes at room temperature in 3% H₂O₂ diluted in methanol and washed with Tris-buffered saline supplemented with 0.01% triton-X100 (TBST). Avidin/biotin blockade was performed using a blocking kit for 15 minutes at room temperature (Abcam, Cambridge, MA), then Fc-receptors were blocked in Fc-blocking solution for 10 minutes at room temperature (BD, San Jose, CA). Sections were further blocked with 3% goat serum in TBST for 30 minutes at room temperature. To stain the tissue, 0.5 μg/ml of AvFc in TBST supplemented with 1% goat serum was added for 30 minutes at room temperature, followed by a biotinylated anti-human IgG in TBST with 1% goat serum also incubated for 30 minutes at room temperature (Vector Labs, Burlingame, CA). Then, ABC solution was added for 30 minutes at room temperature (Vector Labs, Burlingame, CA) followed by the DAB stain, which was applied following the manufacturer's protocol (Vector Labs, Burlingame, CA). Between each step, sections were washed 3 times with TBST. Counter staining was performed with hematoxylin. Sections were serially dehydrated with 95% ethanol, 100% ethanol, and CitriSolv (Decon lab, King of Prussia, PA). Images were taken using an OLYMPUS CKX41 microscope with UPlanFL 10×/0.30 lens (OLYMPUS, Tokyo, Japan).

Statistical Analyses

Group means and standard errors were derived from the values obtained in three individual replicates, and assays were performed at least twice independently unless otherwise noted. For all data, outliers were determined by statistical analysis using the Grubb's test (p<0.05) and excluded from further analysis. Statistical significance was analyzed by one-way or two-way analysis of variance (ANOVA) with Bonferroni's post-hoc test or Wilcoxon matched-pairs signed rank test as indicated in figure legends, using GraphPad Prism 5 (San Diego, CA). Differences were considered statistically significant if p<0.05.

### Example 2. AvFc Selectively Recognizes Various Cancer Cell Lines

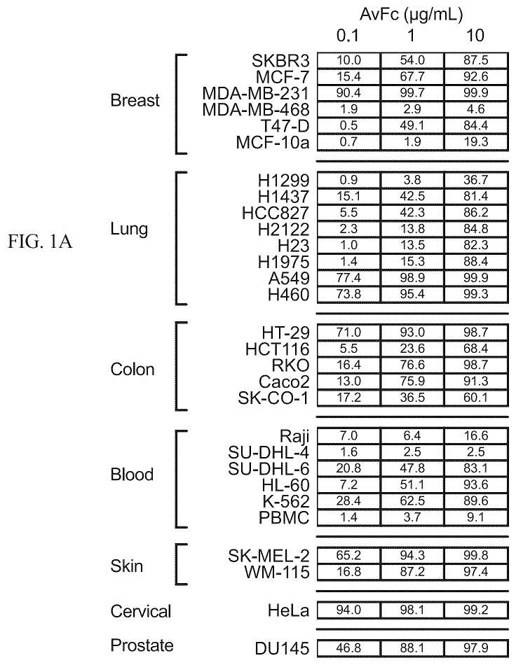

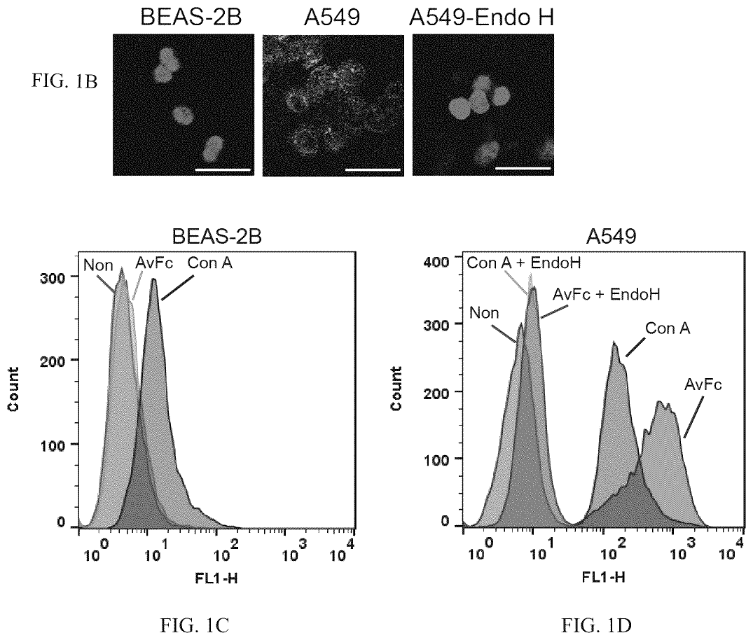

Given that high-mannose glycans are elevated in various neoplastic cells and tissues,^{5-8,10, 13-17}, whether AvFc can effectively recognize cancer cells was tested. Flow cytometry analysis showed that the lectibody bound to a range of human cancer cell lines derived from breast, lung, colon, blood, cervical, and prostate tumors at nanomolar concentrations. Nanomolar concentrations (0.1-10 μg/mL) of AvFc exhibited distinct binding to most of the 27 cancer cell lines tested, albeit with varying degrees of efficiency. MDA-MB-231 breast carcinoma, A549 lung adenocarcinoma, H460 large-cell lung carcinoma, HT-29 colon adenocarcinoma, SK-MEL-2 melanoma, and HeLa cervical carcinoma cell lines were among those most prominently recognized by the lectibody even at the lowest concentration (i.e., 0.1 μg/mL or 1.3 nM) analyzed (FIG. 1A). By contrast, AvFc poorly recognized normal human PBMCs and non-tumorigenic cell lines including MCF10 mammary gland epithelial and BEAS-2B lung epithelial cells. Marginal binding was also noted for relatively few cancer cell lines, including MDA-MB-468 breast carcinoma, Raji Burkitt's lymphoma and SU-DHL-4 B cell lymphoma cells (FIGS. 1A-1D).

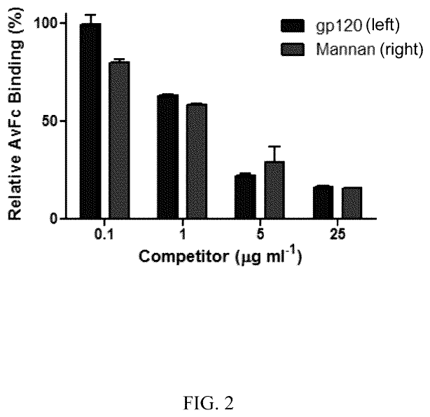

When A549 cells were treated with endoglycosidase H (Endo H), which specifically cleaves high-mannose glycans,²³ the binding of AvFc to the cell line was almost completely abolished (FIGS. 1B-1D). Additionally, the lectibody's binding to A549 cells was dose-dependently inhibited by yeast mannan and the HIV-1 envelope glycoprotein gp120 (FIG. 2). These results demonstrate that AvFc's interaction with cancer cells is mediated via the high-mannose-binding activity of the lectibody's lectin domain. The mannose-binding lectin concanavalin A (Con A) also strongly recognized A549 cells, and similarly to AvFc, this interaction was disrupted by Endo H digestion of cell-surface glycans. Unlike AvFc, however, Con A exhibited a relatively weak yet appreciable degree of interaction with the non-tumorigenic BEAS-2B cells (FIGS. 1C-1D), highlighting distinct glycan recognition mechanisms between the lectibody and the canonical legume lectin.

These analyses indicated that AvFc has high selectivity to malignant cells over noncancerous or normal healthy cells, since AvFc did not show any significant binding to nontumorigenic epithelial cell lines MCF10a and BEAS-2B, human PBMCs (FIGS. 1A-1D), or primary mesenteric lymph node cells isolated from rhesus macaques.¹⁹ AvFc's specificity to high-mannose glycans has been previously demonstrated by a glycan array analysis using over 600 mammalian N-glycans (see FIG. 2B in Hamorsky et al.),¹⁹ strongly indicating the abnormal accumulation of high-mannose glycans on the surface of cancer cells. Similar results regarding the selectivity for cancer-associated high-mannose glycans were previously reported with TM10, an IgM monoclonal antibody isolated from mice immunized with FasL-expressing B16F10 mouse melanoma cells.³⁷ Similar to AvFc, the epitope of TM10 appeared to be clusters of high-mannose glycans, in particular Man9, and the antibody recognized human melanoma, prostate, ovarian, and breast cancer cells with no apparent surface binding to untransformed cells. However, unlike AvFc, TM10 showed little in vivo or in vitro anticancer activity. The inventors attributed the lack of therapeutic effects to the specific isotype of TM10 antibody given that antibodies of IgM isotype typically have poor tissue penetration, short biological half-lives, and lack Fc-mediated effector functions.³⁷ Thus, while it appears that selectivity for cancer cells is similar between AvFc and TM10, the presence of the Fc region from IgG1 is the major differentiating factor of AvFc, a molecular design that offers significant advantages as a potential anti-cancer agent.

These findings add to growing evidence indicating that a high proportion of high-mannose glycans represents a unique characteristic of the cancer cell glycocalyx. Unlike other conventional mannose-binding lectins like Con A, AvFc preferentially recognizes clusters or groupings of high-mannose glycans containing terminal α1,2-linked mannose residues.^{19,22} Such a high density of high-mannose structures is rare in the glycocalyx of normal cells, as illustrated by the data showing AvFc's inability to recognize the nontumorigenic BEAS-2B bronchial epithelial cell line while Con A showed noticeable binding (FIGS. 1C-1D). Con A interacts with both internal and external α-D-mannosyl and α-D-glucosyl residues and has four sugar-binding sites.³⁸ As such, it is more “promiscuous” than AvFc and capable of recognizing a broader spectrum of glycoforms. Furthermore, additional data suggest that AvFc has low affinity to individual glycans and glycoproteins with small numbers of glycans but high affinity to high-mannose rich glycoproteins like HIV gp120 (not shown). This implies that there exists a threshold level of high-mannose glycans that must be present in proximity in order for AvFc to bind with any appreciably affinity, and non-cancer cells simply may not reach this threshold. The data herein support the notion that AvFc is superior to conventional mannose-specific lectins with respect to selectivity to tumor-associated high-mannose glycans.

### Example 3. AvFc Binds to EGFR and IGF1R and Blocks their Signaling

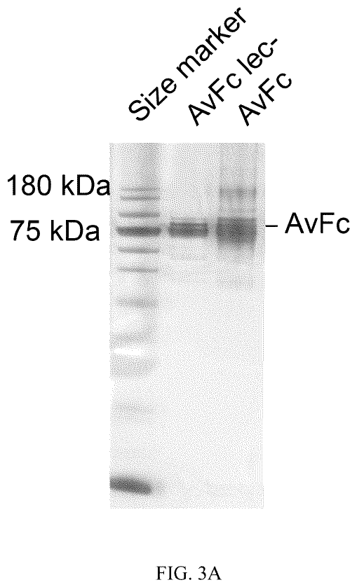

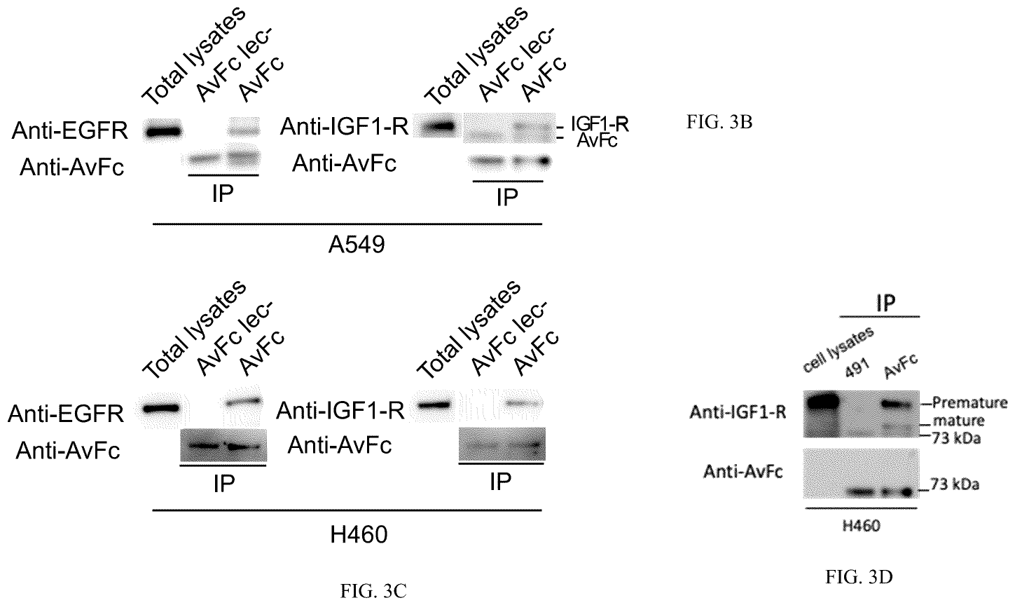

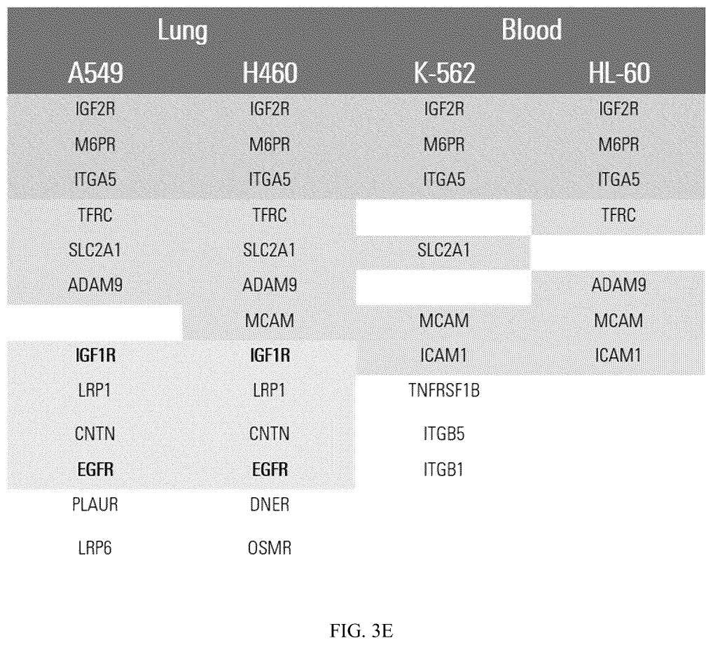

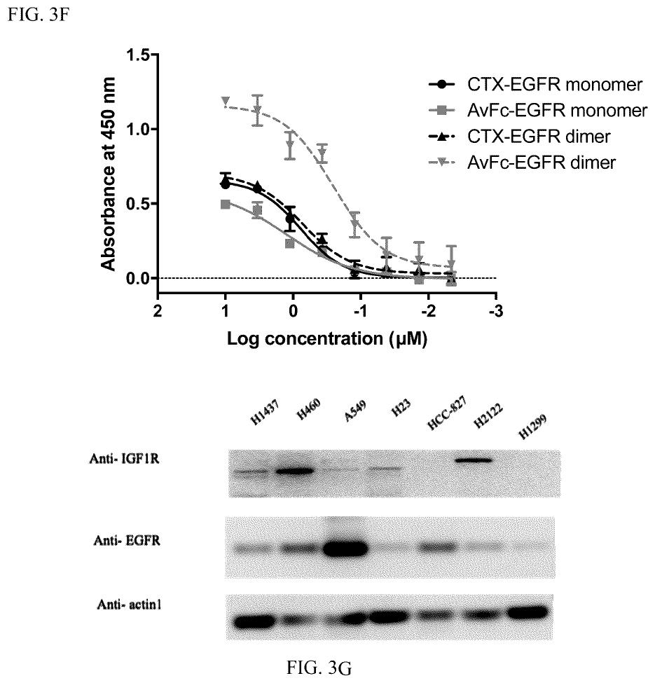

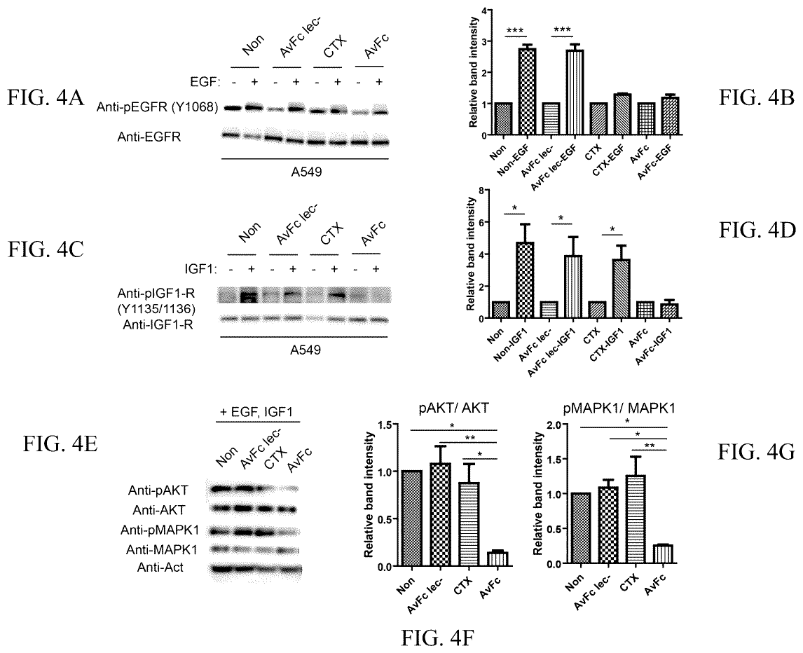

The investigation continued with A549 and H460 cells, two representative NCSLC cells lines that exhibited high AvFc binding in the flow cytometry analysis (FIG. 1A), with a half-maximal effective concentration of approximately 42 ng/mL for A549 and 30 ng/mL for H460. To identify the molecular targets of AvFc in these cells, a pull-down assay was employed using Protein A beads conjugated with AvFc or AvFc^{lec-}, the latter of which is a variant of the lectibody lacking high-mannose binding activity.²² Binding partners were isolated from A549 and H460 cell lysates and identified using mass spectrometry. Silver staining revealed unique proteins in the AvFc-bound fraction that were not isolated by the negative control, AvFc^{lec-} (FIG. 3A). Proteomics analyses of these fractions showed that AvFc recognized a large number of molecules that are found on the cell surface and in the extracellular matrix (Table 2), with many of these being common between the two NSCLC cell lines. These included laminins, integrins, transporters and growth factor receptors (Table 1). Two major growth factor receptors, epidermal growth factor receptor (EGFR) and insulin-like growth factor 1 receptor (IGF1R) were focused on, as they are known to play pivotal roles in cancer progression in NSCLC.²⁴⁻²⁶ To validate the proteomics results, co-immunoprecipitation immunoblot analysis was performed; the results confirmed interaction between AvFc and these receptors in both A549 and H460 cells (FIGS. 3B-D). Because EGFR and IGF1R are dimerized upon ligand binding and phosphorylated to trigger pro-survival signaling cascades,²⁷⁻³⁰, whether AvFc can repress the onset of signal transduction by these receptors was investigated. After pre-incubation with AvFc, AvFc^{lec-} or the FDA-approved anti-EGFR monoclonal antibody cetuximab (CTX) in serum-free medium, A549 cells were treated with EGF or IGF1, and their respective receptors' phosphorylation status were analyzed by immunoblot (FIGS. 4A-4D). The results indicated that AvFc, but not AvFc^{lec-}, blocked the activation of both EGFR and IGF1R as evidenced by a decrease in band intensity of the phosphorylated forms of these receptors (pEGFR and pIGF1R). In contrast, while CTX blocked the activation of EGFR as effectively as AvFc, the EGFR-specific monoclonal antibody failed to inhibit that of IGF1R. To test whether AvFc can simultaneously block these receptors, the inhibition of major downstream signaling pathways shared between EGFR and IGF1R after treating A549 cells was assessed with a mixture of EGF and IGF1, specifically AKT and MAPK pathways that are involved in cell invasion, proliferation and drug resistance.³¹⁻³⁴ Immunoblot analysis showed that AvFc significantly blunted the phosphorylation of AKT and MAPK kinase 1 (MAPK1) upon EGF and IGF1 co-treatment, whereas CTX and AvFc^{lec-} failed to show any inhibition (FIGS. 4E-4G).

TABLE 1
Molecular Targets in Lung Cancers
(shared between A549 and H460)

| UniProt | Gene | |
|---|---|---|
| Accession | Name | Protein Name |
| P00533 | EGFR | Epidermal growth factor receptor |
| Q14517 | FAT1 | Protocadherin FAT1 |
| P08069 | IGF1R | Insulin-like growth factor 1 receptor |
| P17301 | ITGA2 | Integrin alpha-2 |
| P18084 | ITGB5 | Integrin beta-5 |
| P26006 | ITGA3 | Integrin alpha-3 |
| Q07954 | LRP1 | Low density lipoprotein receptor-related |
| | | protein 1 |
| Q9Y666 | SLC12A7 | Solute carrier family12 member 7 |
| P56199 | ITGA1 | Integrin alpha-1 |
| Q9NV96 | TMEM30A | Cell-cycle control protein 50A |
| Q15758 | SLC1A5 | Neutral amino acid transporter B(0) |
| Q9UNN8 | PROCR | Endothelial protein C receptor |
| P07942 | LAMB1 | Laminin subunit beta-1 |
| O15230 | LAMA5 | Laminin subunit alpha-5 |
| P55268 | LAMB2 | Laminin subunit beta-2 |
| O00468-3 | AGRN | Agrin |

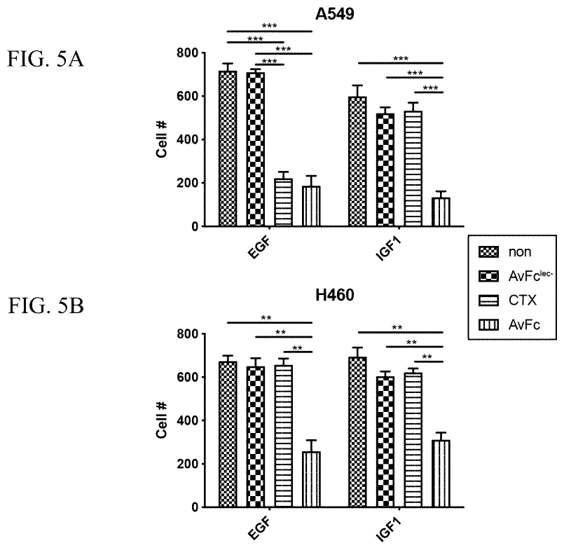

Binding of AvFc to EGFR and IGF1R was dependent on AvFc's lectin activity based on the use of a non-sugar-binding mutant, AvFc^{lec-}, which did not recognize either receptor (FIGS. 3A-3G, 4A-4G and 8A-8D). The lack of high-mannose-binding activity of AvFc^{lec-} has also been clearly demonstrated (see FIG. 5 in Dent et al.²²). The presence of high-mannose glycans on cell-surface EGFR glycans in human cancer has been previously demonstrated by Johns et al.⁵⁵ Similarly, Sato et al. has shown that high-mannose-binding Pseudomonas fluorescence lectin binds to cell surface EGFR in MKN28 gastric cancer cells.⁵⁶ Hasegawa et al. demonstrated that protein D and concanavalin A, which are known as high-mannose binders, recognize EGFR in A549.⁵⁷ Taken together, the data and previously published studies strongly suggest that EGFR and IGF1R in A549 and H460 carry high-mannose glycans.

Given that changes in N-glycosylation modification would globally give rise to any glycoproteins in cancer cells, it is not surprising that the proteomics analysis revealed a large number of proteins recognized by AvFc in A549 and H460 cells (Table 1). Of interest is that EGFR and IGF1R were among the common cell-surface glycoproteins targeted by AvFc in these NSCLC cells (FIGS. 3A-3D). The result indicates that these receptors, which are often overexpressed and strongly associated with cancer progression in NSCLC,²⁴⁻²⁶ display dense high-mannose glycans on the cancer cells. In fact, they are both highly glycosylated, containing as many as 13 and 16 N-glycosylation sites (UniProtKB: P00533 and P08069), respectively. Also, since the ectodomains of EGFR in its activated form and IGF1R exist as dimer,³⁹⁻⁴¹ it is plausible that AvFc exhibits high affinity for these receptors. It was found that AvFc has higher affinity to EGFR dimer than to EGFR monomer (FIG. 3F), which suggests that high expression and activation of EGFR on some cancer cell surfaces may increase local high-mannose glycan concentrations and facilitate AvFc's binding and antitumor activity. One of the consequences of AvFc binding to EGFR and IGF1R was the blockade of their activation and subsequent downstream signaling, as demonstrated by the data showing that AvFc inhibited the phosphorylation of both receptors as well as AKT and MAPK1 upon stimulation with their respective ligands in A549 cells (FIGS. 4A-4G).

The cell lines tested were chosen to represent a broad selection of cancer cell lines that are commercially available, not based on their EGFR or IGF1R status. Testing the binding by flow cytometry (FIGS. 1A-1D) was performed before investigating any potential glycoprotein binding partners on the cell surface (FIGS. 3A-3D). The primary binding mechanism of AvFc to cancer cells is through the overabundance of high-mannose type glycans, which presumably occurs in many if not all glycoproteins that traverse the endomembrane secretory pathway within cancer cells. Thus, although EGFR and IGF1R appear to significantly contribute to the overall AvFc binding level in some cell lines (such as A549 and H460), it is believed that the overall binding to cancer cell lines is not necessarily always dependent on these receptors. This notion is supported by the proteomic analysis (Table 1 and Table 2), which showed a number of other AvFc targets besides EGFR and IGF1R.

The levels of EGFR and IGF1R in some of the lung cancer cell lines were analyzed (FIG. 3G). While A549 and H460 showed the highest level of EGFR and IGF1R, respectively, the levels of these receptors were not noticeably different in the other cell lines, including H1437, which showed the next highest AvFc binding after A549 and H460 (FIG. 1A). This suggests that there appears to be weak, if any, association between EGFR/IGF1R expression levels and AvFc binding. The data also supports that other glycoprotein targets (such as those identified in the proteomics analysis) have contributed to AvFc's recognition of cancer cells. Given that AvFc's binding activity is not solely dependent on a specific receptor, it is believed that the present study highlights the versatility of high-mannose glycan targeting as a novel anti-cancer strategy.

### Example 4. AvFc Inhibits Migration of A549 and H460 Cells

The above results indicate that AvFc effectively binds to both EGFR and IGF1R on the cell surface, thereby intercepting their ligands and preventing receptor activation and subsequent AKT and MAPK signaling. Because AKT and MAPK signaling pathways are involved in migration,^{35, 36}, the effects of AvFc on cell migration using A549 and H460 cells were investigated using a transwell culture system, wherein cells were co-incubated with the lectibody and subsequently treated with EGF or IGF1 in serum-free medium. These cells were then seeded into transwells and after 6 h cells in the bottom chamber were quantified. As shown in FIGS. 5A-5B, AvFc significantly blocked the migration of both cell lines regardless of which growth factor the cells were stimulated with, whereas AvFc^{lec-} failed to show any effect. CTX, on the other hand, only effectively inhibited the migration of A549 cells treated with EGF, but not when the cells were treated with IGF1 (FIG. 5A). Additionally, CTX failed to show any effect on the migration of H460 cells, even when the cells were stimulated with EGF (FIG. 5B).

### Example 5. AvFc Induces ADCC Against Cancer Cells

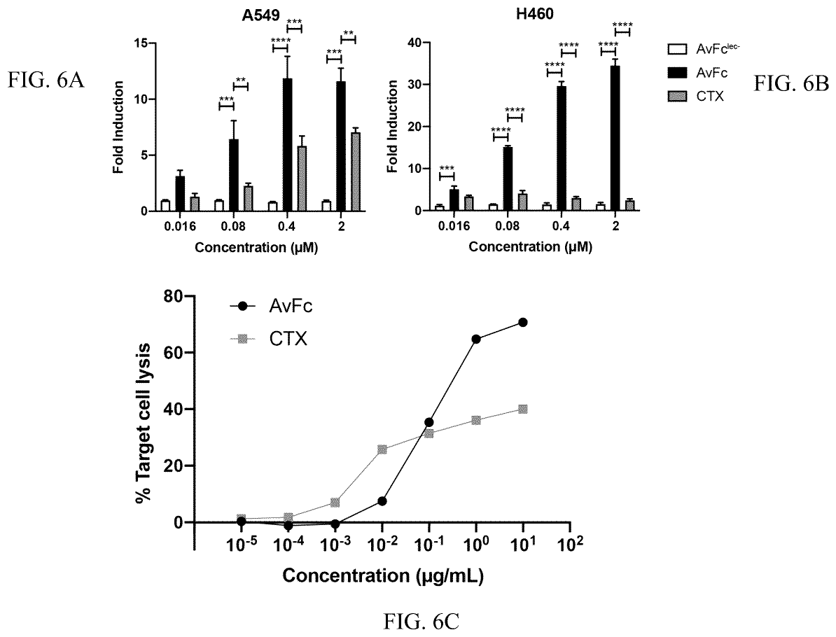

Next, the consequences of AvFc's binding to cancer cells was investigated from a different angle, i.e., whether the Fc region of the lectibody can direct antibody-dependent cell-mediated cytotoxicity (ADCC). Since ADCC is mediated primarily through NK cells expressing FcγRIIIa, the Fc-mediated activity of AvFc against A549 and H460 cells was assessed using an in vitro assay based on FcγRIIIa-activated luciferase expression. As shown in FIGS. 6A-6B, AvFc activated FcγRIIIa in a dose dependent manner. AvFc^{lec-}, on the other hand, failed to activate FcγRIIIa at all concentrations tested, demonstrating that the activity is dependent upon AvFc's binding to high-mannose glycans on cancer cells. Of note, AvFc showed significantly higher efficacy against A549 cells than CTX at the top three concentrations tested (0.08, 0.40 and 2.00 μM). Moreover, AvFc exhibited remarkable activity (a maximum over 30-fold above baseline) against H460, while CTX was ineffective for the large-cell lung carcinoma cell-line (FIGS. 6A-6B). To confirm the ability of AvFc to induce Fc-mediated cell killing activity, a canonical ADCC assay was performed using human PBMC effector cells and A549 cells as the targets. As shown in FIG. 6C, the lectibody showed a dose-dependent cell lysis activity against A549 cells; it is of note, however, that the efficacy of the lectibody was nearly twice as high as that of CTX (˜80% for AvFc vs. ˜40% for CTX).

### Example 6. AvFc Exhibits Antitumor Effects in Mouse A549 and H460 Xenograft Models

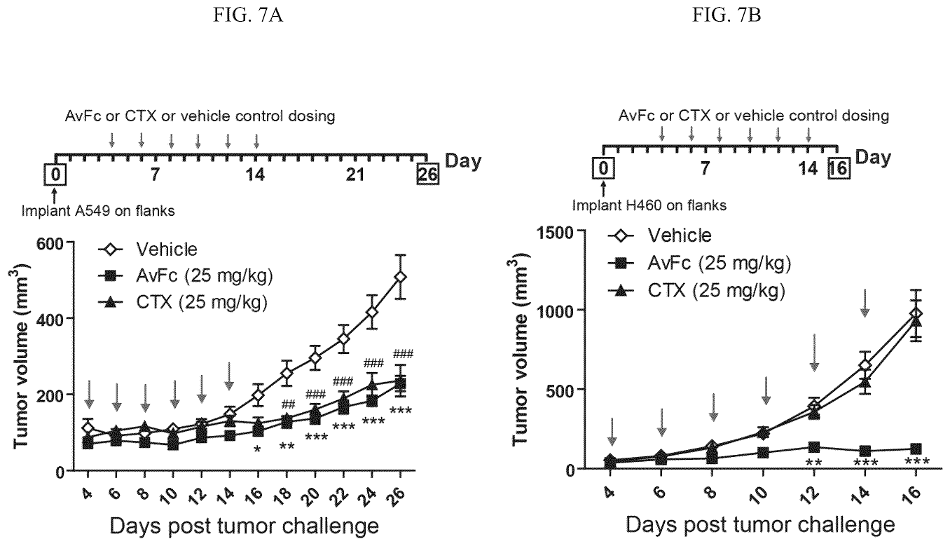

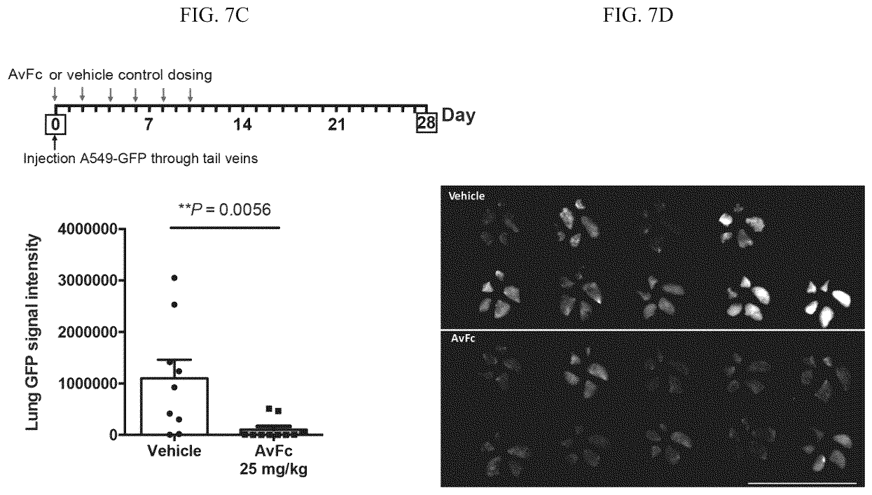

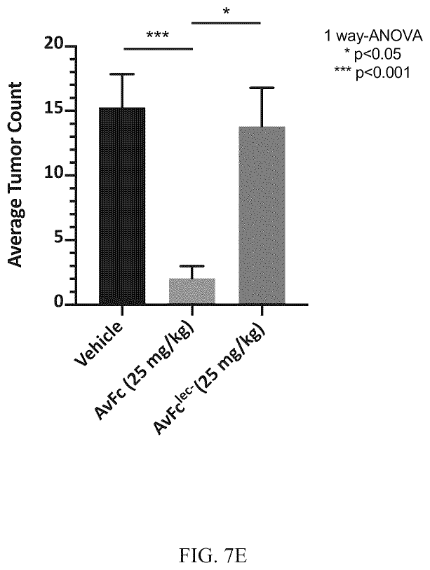

The anti-tumor effects of AvFc were evaluated in Prkdc^{scid}/SzJ (SCID) mice challenged with A549 and H460 xenografts implanted in the hind left flank. Intraperitoneal treatment with 25 mg/kg of AvFc or CTX was initiated at day 4 post tumor challenge and continued every two days for a total of 6 doses. AvFc treatment significantly blunted A549 (FIG. 7A) and H460 (FIG. 7B) tumor growth compared to the vehicle control. On the other hand, CTX showed similar efficacy to AvFc against A549 tumors but failed to show an effect on the growth of H460 tumors. To further evaluate AvFc's anti-tumor effect, mice were intravenously challenged with A549-GFP and subsequently treated with the same dosing regimen as in the flank tumor models. Fluorescence imaging of the lung isolated 18 days after the last dose showed that AvFc significantly inhibited the growth of A549-GFP cells in the lung as compared to a vehicle control (FIGS. 7C-7D). Taken together, these data clearly demonstrated that AvFc has the ability to elicit antitumor activity in vivo.

To further elucidate the potential anti-tumor mechanism of action of AvFc, ADCC activities were measured in both reporter cell-based assays and human PBMC-based assays (FIGS. 6A-6C). In both assay formats, AvFc elicited a strong ADCC response by effectively activating FcγRIIIa on the surface of engineered Jurkat cells (which express luciferase in response to activation) and PBMCs. Conversely, CTX had no activity against H460 cells and only moderate activity against A549 in both the reporter cell assay and PBMC-based assay, underperforming AvFc. This was consistent with the anti-tumor effects of AvFc seen in PrkdC^{scid}/SzJ (SCID) mice challenged with A549 and H460 xenografts (FIGS. 7A-7D), wherein AvFc treatment slowed the growth of both xenografts while CTX was only efficacious against A549. Taken together, these results suggest that binding and inhibition of multiple cell-surface receptors in addition to more potent ADCC activity resulted in AvFc's superior anticancer activity in these models.

### Example 7. AvFc Preferentially Binds to Human NSCLC Tumor Tissue

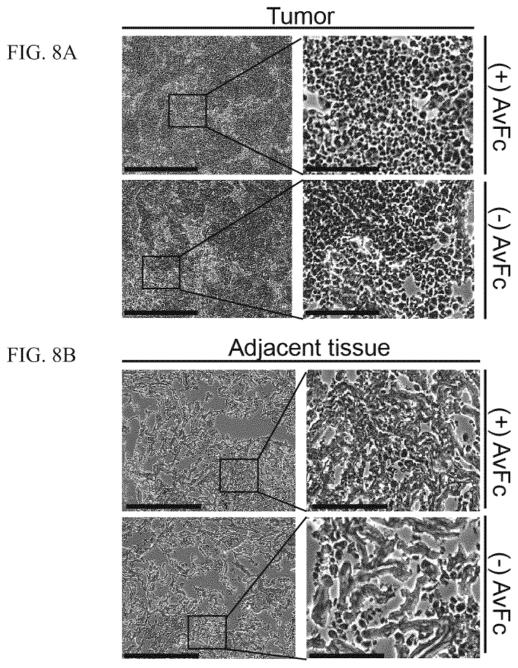

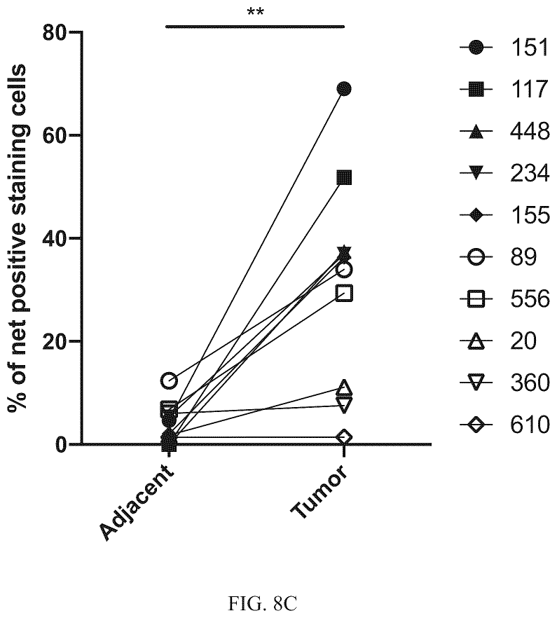

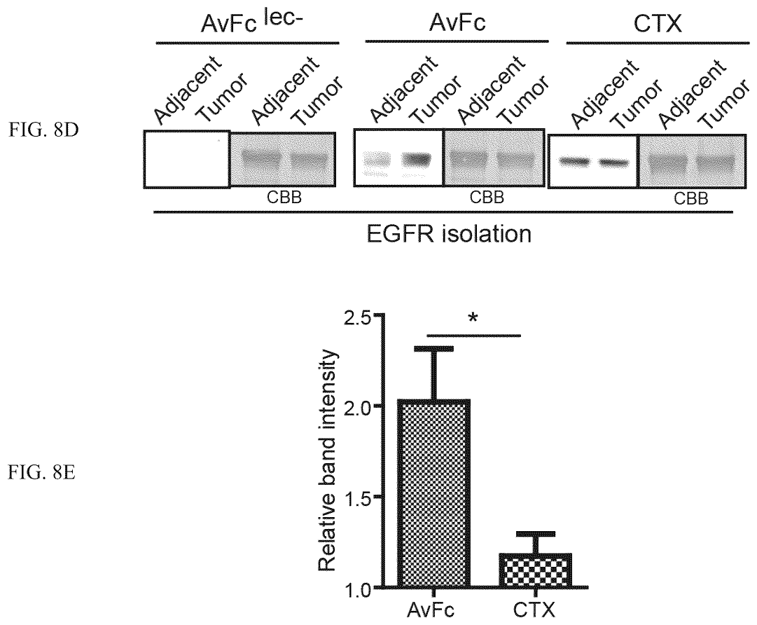

The binding of AvFc to primary tumor and adjacent tissues isolated from human NSCLC patients was investigated using immunohistochemistry (IHC). Compared to the adjacent tissue, AvFc binding was more evident in NSCLC tumor (FIGS. 8A-8B), indicating that the lectibody is capable of distinguishing their differential glycosylation patterns. Among the matched pair tissues from 10 NSCLC patients analyzed, 7 showed significantly higher AvFc binding in tumors than in the adjacent tissue (FIG. 8C). Given that EGFR was one of the major molecular targets of AvFc in A549 and H460 (FIGS. 3A-3D), it was postulated that AvFc's tumor selectivity found in NSCLC patients' lung tissues may be partly attributed to the receptor. Thus, EGFR was enriched from the tumor and adjacent tissue lysates from five NSCLC patients (sample No. 151, 117, 448, 234, 155) using co-immunoprecipitation and then detected with AvFc, CTX, or AvFc^{lec-} by Western blot. A representative image of tissue samples from Patient 117 is shown in FIG. 8D, and relative band intensities between tumor EGFR and the adjacent tissue-derived counterpart are shown in FIG. 8E. Although AvFc reacted with both tumor and adjacent tissue EGFRs, it showed a stronger signal with the former (approximately 2-fold on average; FIG. 8E), indicating that the lectibody had higher affinity to tumor-derived EGFR. By contrast, CTX showed bands of similar intensity for tumor and adjacent tissue EGFRs in all five tissues tested (FIG. 8D-8E). AvFc^{lec-} failed to probe the receptor from both tumor and adjacent tissues. Taken together, these results indicate that AvFc has preferential binding to NSCLC tumor-derived EGFR over that of the normal lung tissue, while CTX cannot distinguish them.

AvFc's ability to inhibit both EGFR and IGF1R has important therapeutic implications, as currently there is no FDA-approved anticancer drug that can simultaneously block these receptors. CTX, a FDA-approved anti-EGFR antibody therapeutic used in the present study as a reference, was only able to block EGFR but not IGF1R in A549 (FIGS. 4A-4G and 5A-5B) and, in stark contrast to AvFc, could not exhibit any antitumor effect against the H460 cell line (FIGS. 5A-5B, 6A-6B and 7A-7C), which is known to be CTX resistant.⁴² The remarkable in vivo antitumor efficacy observed with AvFc is most likely due to its effective binding to high-mannose glycans that are broadly and highly accumulated on the surface of cancer cells; in our ongoing study, AvFc showed a similar antitumor effect protecting against B16F10 melanoma lung metastasis while the non-sugar-binding mutant AvFc^{lec-} failed to show any effect (FIG. 7E). Despite the fact that colorectal cancer patients show significant improvements in response rates, overall survival and progression-free survival after CTX treatment, CTX responses in NSCLC patients have not been convincing in clinical trials.⁴³⁻⁴⁵ IGF1R may be involved in the resistance mechanism of CTX and other EGFR-targeted drugs,⁴⁶⁻⁴⁹ since these two receptors share similar downstream signaling pathways (PI3K/AKT/MAPK/NF-κB); IGF1R can bypass EGFR inhibition, while their cooperation may promote tumor growth and progression.⁴⁸⁻⁵⁰ One study revealed that overexpression of both EGFR and IGF1R was observed in 24.8% of 125 surgical NSCLC patients, and high co-expression of EGFR and IGF1R was a significant prognostic factor of worse disease-free survival.⁵⁰

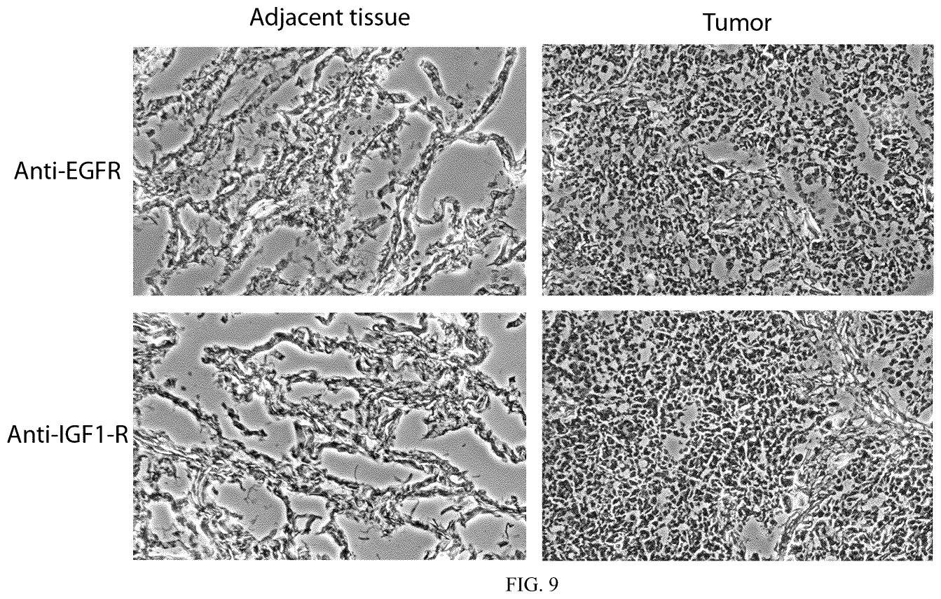

The selectivity of AvFc was evaluated in 10 pairs of tumor and adjacent lung tissues from NSCLC patients (FIGS. 8A-8D). Overall, AvFc interacted preferentially with tumor tissue and was capable of distinguishing the tumor and adjacent tissues, despite the low level of background AvFc staining in the latter. High-mannose expression levels within the tissues likely depend on the developmental stage of the cancer. For example, IHC analysis showed that tumors express more EGFR and IGF1R than the adjacent normal tissues (FIG. 9), supporting the higher binding of AvFc to tumors. The selective interaction of AvFc with primary NSCLC cells in this analysis demonstrates the utility of AvFc beyond animal models and suggests that it may be able to effectively target tumors in NSCLC patients.

Aberrant glycosylation has long been recognized as a hallmark of cancer. Nevertheless, development of therapeutics targeting cancer-associated glycans has been slow. Examples 2-7 showed that AvFc, a lectibody specific to high-mannose glycans,¹⁹ can recognize multiple human cancer cell lines derived from various cancer types. The therapeutic implications of AvFc's interaction with cancer cells were evaluated using two NSCLC cell lines A549 and H460, demonstrating that the lectibody can block the activation of EGFR and IGF1R and cell migration upon stimulation with their respective ligands, elicit ADCC activity, and significantly delay xenograft tumor growth in SCID mice. Furthermore, IHC analysis showed that AvFc preferentially binds to primary human NSCLC tumors over adjacent non-tumor lung tissues isolated. To the Inventor's knowledge, this is the first report demonstrating the antitumor effects of an agent targeting cancer-associated high-mannose glycans.

These findings lend support for the notion that high-mannose glycans constitute a cancer glycobiomarker. It remains elusive how or why high-mannose glycans become over-represented on the surface of cancer cells in the first place. A few cellular mechanisms have been identified which allow the accumulation of immature glycans in tumors, including stress-independent activation of X-box binding protein 1 (XBP1), misregulation of N-acetylglucosaminyltransferase, and downregulation of α-mannosidase I and mannosyl(α-1,3-)-glycoprotein 0-1,2-N-acetylglucosaminyltransferase (MGAT1).^{11, 12, 51, 52} Stress-independent activation of XBP1, for instance, was found to reduce sialylation and bisecting GlcNAc while increasing the levels of high-mannose glycans in HEK293 and HeLa cells by affecting mannosidase expression.⁵¹ The significant reduction of α-mannosidase I expression found in cholangiocarcinoma cells was correlated to the elevation of high-mannose glycans and a more metastatic phenotype.¹² The use of kifunensine, a small-molecule inhibitor of α-mannosidase I, also increased high-mannose glycan content and produced similar results.¹² Takayama et al. have shown that increased high-mannose glycan expression, detected in surgical specimens of hepatocellular carcinoma, was associated with decreased expression of MGAT1, a key glycosyltransferase that converts high-mannose glycans to complex- or hybrid-type N-glycans.¹¹ Meanwhile, high-mannose glycans at the helical domain of transferrin receptor protein 1 appear to trigger structural changes that improve noncovalent interaction energies, resulting in cell migration enhancement in metastatic cholangiocarcinoma.¹² A recent publication assessing the impact of high-mannose glycans on bone-marrow-derived mesenchymal stromal cells has provided evidence that these glycans alter the physical and structural properties of the cells themselves, decreasing their size and increasing motility, which may in part explain the greater metastatic potential seen in other cell lines.⁵³ Given the growing body of evidence indicating the close association between the abundance of high-mannose glycans on cancer cells and increased cell migration and metastatic potential, it is of high clinical significance to uncover the cause and process leading to high-mannose overexpression in cancer and to scrutinize its functions in tumor microenvironments and metastasis. In this regard, AvFc may provide a valuable tool to probe and monitor high-mannose glycan accumulation on cell surface, thereby facilitating such investigations.

In summary, Examples 2-7 demonstrated that AvFc, a lectibody targeting high-mannose glycans, can selectively recognize cancer cells and exert antitumor activity possibly through a combination of growth factor receptor inhibition and immune activation via Fc receptors. These findings suggest that increased abundance of high-mannose glycans in cancer, such as those expressed on EGFR and IGF1R, present druggable targets in NSCLC, and AvFc provides a new tool to probe and target tumor-associated high-mannose glycan biomarker.

The immunogenicity and toxicity of drug candidates are critical parts of drug development. Previous studies showed that AvFc did not cause any significant toxicity in rats or rhesus macaques after a single dose or mice after repeated dosing.¹⁹ Additionally, a recent publication also showed that repeated systemic dosing at 25 mg/kg up to 11 times over 20 days did not result in any noticeable toxicity while protecting against a hepatitis C virus challenge in human liver chimeric mice.²²

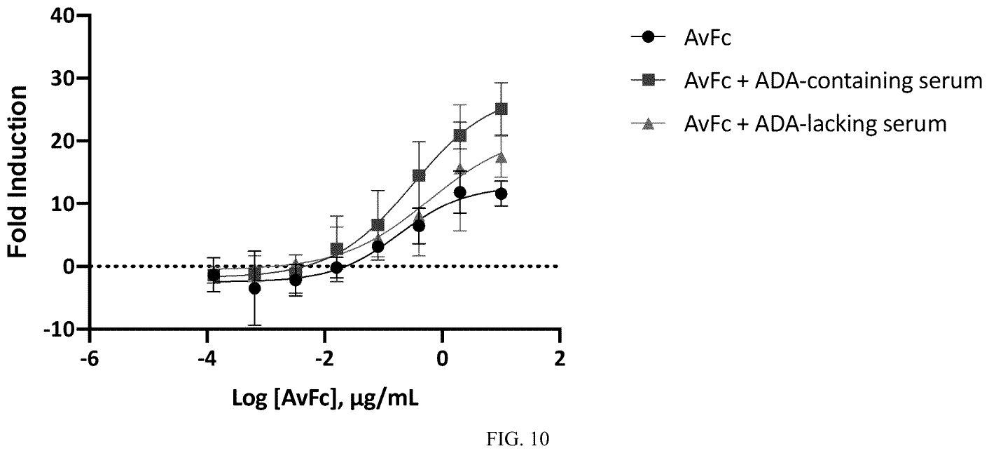

Regarding immunogenicity, AvFc were produced in a glycoengineered plant host devoid of plant-specific N-glycoforms (K1DFX-P2 N. benthamiana) as previously described,^{19,54} thus avoiding the potential immunogenicity due to plant-specific glycans. AvFc induced an anti-drug antibody (ADA) response in mice, however, the response could be due (at least in part) to the human IgG Fc domain. An in vitro ADCC assay showed that the pooled mouse serum containing AvFc ADAs failed to inhibit (rather slightly increased) the ADCC activity of AvFc against A549 cells, suggesting that ADAs may not have significant impact on the lectibody's antitumor activity, if any (FIG. 10).

Sequences

SEQ ID NO:1 is an amino acid sequence of a wild type actinohivin polypeptide

(SEQ ID NO: 1)
ASVTIRNAQTGRLLDSNYNGNVYTLPANGGNYQRWTGPGDGTVRNAQTG
RCLDSNYDGAVYTLPCNGGSYQKWLFYSNGYIQNVETGRVLDSNYNGNV
YTLPANGGNYQKWYTG

SEQ ID NO:2 is an amino acid sequence of an actinohivin variant polypeptide made in accordance with the presently-disclosed subject matter and designated herein as variant 1.

(SEQ ID NO: 2)
ASGTIRNAETGRLLDSNYDGAVYTLPANGGSYQRWTGPGDGTVRNAETG
RLLDSNYDGAVYTLPANGGSYQKWTGPGDGTIQNAETGRLLDSNYDGAV
YTLPANGGSYQKWTG

SEQ ID NO:3 is an amino acid sequence of another actinohivin variant polypeptide made in accordance with the presently-disclosed subject matter and designated herein as variant

(SEQ ID NO: 3)
ASGTIRNAETGRCLDSNYDGAVYTLPCNGGSYQRWTGPGDGTVRNAETG
RCLDSNYDGAVYTLPCNGGSYQKWTGPGDGTIQNAETGRCLDSNYDGAV
YTLPCNGGSYQKWTG

SEQ ID NO:4 is an amino acid sequence of another actinohivin variant polypeptide made in accordance with the presently-disclosed subject matter and designated herein as variant 3.

(SEQ ID NO: 4)
ASVTIRNAETGRLLDSNYNGNVYTLPANGGNYQRWTGPGDGTVRNAETG
RCLDSNYDGAVYTLPCNGGSYQKWLFYSNGYIQNVETGRVLDSNYNGNV
YTLPANGGNYQKWYTG

SEQ ID NO:5 is an amino acid sequence of another actinohivin variant polypeptide made in accordance with the presently-disclosed subject matter and designated herein as variant 4.

(SEQ ID NO: 5)
ASVTIRNAETGRCLDSNYNGNVYTLPCNGGNYQRWTGPGDGTVRNAETG
RCLDSNYDGAVYTLPCNGGSYQKWLFYSNGYIQNVETGRCLDSNYNGNV
YTLPCNGGNYQKWYTG

SEQ ID NO:6 is an amino acid sequence of another actinohivin variant polypeptide made in accordance with the presently-disclosed subject matter and designated herein as variant 5.

(SEQ ID NO: 6)
ASGTIRNAETGRLLDSNYNGNVYTLPANGGNYQRWTGPGDGTVRNAETG
RCLDSNYDGAVYTLPCNGGSYQKWTGPGDGTIQNAETGRVLDSNYNGNV
YTLPANGGNYQKWTG

SEQ ID NO:7 is an amino acid sequence of another actinohivin variant polypeptide made in accordance with the presently-disclosed subject matter and designated herein as variant 6.

(SEQ ID NO: 7)
ASGTIRNAETGRCLDSNYDGNVYTLPCNGGSYQRWTGPGDGTVRNAETG
RCLDSNYDGNVYTLPCNGGSYQKWTGPGDGTIQNAETGRCLDSNYDGNV
YTLPCNGGSYQKWTG

SEQ ID NO:8 is an amino acid sequence of another actinohivin variant polypeptide made in accordance with the presently-disclosed subject matter and designated herein as variant 7.

(SEQ ID NO: 8)
ASGTIRNAQTGRCLDSNYNGNVYTLPCNGGNYQRWTGPGDGTVRNAQTG
RCLDSNYDGAVYTLPCNGGSYQKWTGPGDGTIQNAETGRCLDSNYNGNV
YTLPCNGGNYQKWTG

SEQ ID NO:9 is an amino acid sequence of another actinohivin variant polypeptide made in accordance with the presently-disclosed subject matter and designated herein as variant 8 or Avaren (actinohivin variant expressed in Nicotiana).

(SEQ ID NO: 9)
ASGTIRNAETGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTG

SEQ ID NO: 10 is an amino acid sequence of another actinohivin variant polypeptide made in accordance with the presently-disclosed subject matter and designated herein as variant 9.

(SEQ ID NO: 10)
ASGTIRNAQTGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTG

SEQ ID NO:11 is an amino acid sequence of another actinohivin variant polypeptide made in accordance with the presently-disclosed subject matter and designated herein as variant 10.

(SEQ ID NO: 11)
ASGTIRNAETGRCLDSNYNGNVYTLPCNGGNYQRWTGPGDGTVRNAQTG
RCLDSNYDGAVYTLPCNGGSYQKWTGPGDGTIQNAETGRCLDSNYNGNV
YTLPCNGGNYQKWTG

SEQ ID NO: 12 is an amino acid sequence of another actinohivin variant polypeptide made in accordance with the presently-disclosed subject matter and designated herein as variant 11.

(SEQ ID NO: 12)
ASGTIRNAQTGRLLDSNYNGNVYTLPANGGNYQRWTGPGDGTVRNAQTG
RLLDSNYNGNVYTLPANGGNYQKWTGPGDGTIQNAQTGRVLDSNYNGNV
YTLPANGGNYQKWTG

SEQ ID NO: 13 is an amino acid sequence of another actinohivin variant polypeptide made in accordance with the presently-disclosed subject matter and designated herein as variant 12.

(SEQ ID NO: 13)
ASGTIRNAETGRLLDSNYNGNVYTLPANGGNYQRWTGPGDGTVRNAETG
RLLDSNYNGNVYTLPANGGNYQKWTGPGDGTIQNAETGRVLDSNYNGNV
YTLPANGGNYQKWTG

SEQ ID NO: 14 is an amino acid sequence of another actinohivin variant polypeptide made in accordance with the presently-disclosed subject matter and designated herein as variant 13.

(SEQ ID NO: 14)
ASGTIRNAETGRCLDSNYNGNVYTLPCNGGNYQRWTGPGDGTVRNAETG
RCLDSNYNGNVYTLPCNGGNYQKWTGPGDGTIQNAETGRCLDSNYNGNV
YTLPCNGGNYQKWTG

SEQ ID NO: 15 is an amino acid sequence of another actinohivin variant polypeptide made in accordance with the presently-disclosed subject matter and designated herein as variant 14.

(SEQ ID NO: 15)
ASGTIRNAETGRCLDSNYDGNVYTLPCNGGNYQRWTGPGDGTVRNAETG
RCLDSNYDGNVYTLPCNGGNYQKWTGPGDGTIQNAETGRCLDSNYDGNV
YTLPCNGGNYQKWTG

SEQ ID NO: 16 is an amino acid sequence including the actinohivin variant polypeptide of SEQ ID NO:9 (Variant 8) fused, via a linker polypeptide, to an amino acid sequence comprising the fragment crystallizable (Fc) region of immunoglobulin (Ig) G, and referred to herein as AvFc.

(SEQ ID NO:16)
ASGTIRNAETGRCLDSNYNGNVYTLPCNGGNYQRWTGPGDGTVRNAETG
RCLDSNYDGAVYTLPCNGGSYQKWTGPGDGTIQNAETGRCLDSNYNGNV
YTLPCNGGNYQKWTGGGGSVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

TABLE 2
Putative Cell-Surface Binding Partners of AvFc on A549 and H460 Cells.

| | | |
|---|---|---|
| | | Percentage of |
| | | Total Spectra |
| Gene Name | Uniprot Accession | (%) |

Putative A549 cell-surface binding partners

| | | |
|---|---|---|
| Laminin subunit beta-1 | sp\|P07942\|LAMB1_HUMAN | 0.4100 |
| Laminin subunit gamma-1 | sp\|P11047\|LAMC1_HUMAN | 0.3600 |
| Laminin subunit alpha-5 | sp\|O15230\|LAMA5_HUMAN | 0.3400 |
| Agrin | sp\|O00468-3\|AGRIN_HUMAN | 0.2100 |
| Mucin-5AC | sp\|P98088\|MUC5A_HUMAN | 0.2100 |
| Cation-independent mannose-6-phosphate receptor | sp\|P11717\|MPRI_HUMAN | 0.2000 |
| Epidermal growth factor receptor | sp\|P00533\|EGFR_HUMAN | 0.1900 |
| Isoform 6 of Myoferlin | sp\|Q9NZM1-6\|MYOF_HUMAN | 0.1700 |
| Polycystin-2 | sp\|Q13563-3\|PKD2_HUMAN | 0.1700 |
| Transferrin receptor protein 1 | sp\|P02786\|TFR1_HUMAN | 0.1600 |
| Integrin alpha-V | sp\|P06756-2\|ITAV_HUMAN | 0.1500 |
| Erlin-2 | sp\|O94905\|ERLN2_HUMAN | 0.1500 |
| Integrin alpha-3 | sp\|P26006\|ITA3_HUMAN | 0.1500 |
| Mucin-5B | sp\|Q9HC84\|MUC5B_HUMAN | 0.1400 |
| Tetratricopeptide repeat protein 13 | sp\|Q8NBP0\|TTC13_HUMAN | 0.1300 |
| CD109 antigen | sp\|Q6YHK3\|CD109_HUMAN | 0.1300 |
| Tetratricopeptide repeat protein 17 | sp\|Q96AE7-2\|TTC17_HUMAN | 0.1300 |
| Plexin-B2 | sp\|O15031\|PLXB2_HUMAN | 0.1200 |
| Protocadherin Fat 1 | sp\|Q14517\|FAT1_HUMAN | 0.1200 |
| Sortilin-related receptor | sp\|Q92673\|SORL_HUMAN | 0.1200 |
| Transmembrane protein 2 | sp\|Q9UHN6-2\|TMEM2_HUMAN | 0.1100 |
| Integrin beta-5 | sp\|P18084\|ITB5_HUMAN | 0.1100 |
| Spectrin beta chain, non-erythrocytic 1 | sp\|Q01082\|SPTB2_HUMAN | 0.1100 |
| Erlin-1 | sp\|O75477\|ERLN1_HUMAN | 0.1100 |
| Integrin alpha-2 | sp\|P17301\|ITA2_HUMAN | 0.1100 |
| Transmembrane and TPR repeat-containing protein 3 | sp\|Q6ZXV5-2\|TMTC3_HUMAN | 0.1100 |
| Contactin-associated protein-like 3 | sp\|Q9BZ76\|CNTP3_HUMAN | 0.1100 |
| Disintegrin and metalloproteinase domain-containing protein 9 | sp\|Q13443\|ADAM9_HUMAN | 0.1100 |
| Transmembrane protein 106B | sp\|Q9NUM4\|T106B_HUMAN | 0.0980 |
| Insulin-like growth factor 1 receptor | sp\|P08069\|IGF1R_HUMAN | 0.0910 |
| Metal transporter CNNM4 | sp\|Q6P4Q7\|CNNM4_HUMAN | 0.0910 |
| Adhesion G protein-coupled receptor L2 | sp\|095490-2\|AGRL2_HUMAN | 0.0910 |
| T-complex protein 1 subunit theta | sp\|P50990\|TCPQ_HUMAN | 0.0910 |
| Attractin | sp\|O75882\|ATRN_HUMAN | 0.0840 |
| Tropomyosin alpha-3 chain | sp\|P06753-2\|TPM3_HUMAN | 0.0700 |
| Multidrug resistance-associated protein 1 | sp\|P33527-3\|MRP1_HUMAN | 0.0700 |
| Galectin-3-binding protein | sp\|Q08380\|LG3BP_HUMAN | 0.0700 |
| Serpin H1 | sp\|P50454\|SERPH_HUMAN | 0.0630 |
| Prolow-density lipoprotein receptor-related protein 1 | sp\|Q07954\|LRP1_HUMAN | 0.0560 |
| Exportin-1 | sp\|O14980\|XPO1_HUMAN | 0.0560 |
| Disintegrin and metalloproteinase domain-containing protein 17 | sp\|P78536\|ADA17_HUMAN | 0.0560 |
| Importin subunit beta-1 | sp\|Q14974\|IMB1_HUMAN | 0.0560 |
| Transmembrane protein 131 | sp\|Q92545\|TM131_HUMAN | 0.0560 |
| Laminin subunit beta-2 | sp\|P55268\|LAMB2_HUMAN | 0.0560 |
| Scavenger receptor class B member 1 | sp\|Q8WTV0-2\|SCRB1_HUMAN | 0.0560 |
| Sodium/potassium-transporting ATPase subunit beta-1 | sp\|P05026-2\|AT1B1_HUMAN | 0.0560 |
| Integrin alpha-5 | sp\|P08648\|ITA5_HUMAN | 0.0350 |
| Cation-dependent mannose-6-phosphate receptor | sp\|P20645\|MPRD_HUMAN | 0.0280 |
| Solute carrier family 12 member 7 | sp\|Q9Y666\|S12A7_HUMAN | 0.0210 |
| Integrin alpha-1 | sp\|P56199\|ITA1_HUMAN | 0.0210 |
| Cell cycle control protein 50A | sp\|Q9NV96\|CC50A_HUMAN | 0.0210 |
| Magnesium transporter protein 1 | sp\|Q9HOU3\|MAGT1_HUMAN | 0.0210 |
| Neutral amino acid transporter B (0) | sp\|Q15758\|AAAT_HUMAN | 0.0140 |
| Endothelial protein C receptor | sp\|Q9UNN8\|EPCR_HUMAN | 0.0140 |
| Solute carrier family 2, facilitated glucose transporter member 1 | sp\|P11166\|GTR1_HUMAN | 0.0070 |

Putative H460 cell-surface binding partners

| | | |
|---|---|---|
| Laminin subunit gamma-1 | sp\|P11047\|LAMC1_HUMAN | 0.8300 |
| Cation-independent mannose-6-phosphate receptor | sp\|P11717\|MPRI_HUMAN | 0.4400 |
| Laminin subunit alpha-1 | sp\|P25391\|LAMA1_HUMAN | 0.3500 |
| Laminin subunit beta-1 | sp\|P07942\|LAMB1_HUMAN | 0.3100 |
| Laminin subunit alpha-5 | sp\|O15230\|LAMA5_HUMAN | 0.2300 |
| ATP-binding cassette sub-family A member 2 | sp\|Q9BZC7-3\|ABCA2_HUMAN | 0.2100 |
| Fibronectin | sp\|P02751-17\|FINC_HUMAN | 0.2100 |
| Transferrin receptor protein 1 | sp\|P02786\|TFR1_HUMAN | 0.2100 |
| Galectin-3-binding protein | sp\|Q08380\|LG3BP_HUMAN | 0.1700 |
| Laminin subunit beta-2 | sp\|P55268\|LAMB2_HUMAN | 0.1600 |
| Scavenger receptor class B member 1 | sp\|Q8WTV0-2\|SCRB1_HUMAN | 0.1500 |
| Agrin | sp\|O00468-3\|AGRIN_HUMAN | 0.1500 |
| Plexin-B2 | sp\|O15031\|PLXB2_HUMAN | 0.1300 |
| Contactin-associated protein 1 | sp\|P78357\|CNTP1_HUMAN | 0.1300 |
| Integrin alpha-1 | sp\|P56199\|ITA1_HUMAN | 0.1300 |
| CD109 antigen | sp\|Q6YHK3-4\|CD109_HUMAN | 0.1300 |
| Filamin-A | sp\|P21333-2\|FLNA_HUMAN | 0.1300 |
| Integrin alpha-V | sp\|P06756-2\|ITAV_HUMAN | 0.1300 |
| Integrin beta-5 | sp\|P18084\|ITB5_HUMAN | 0.1200 |
| Myoferlin | sp\|Q9NZM1-2\|MYOF_HUMAN | 0.1100 |
| Tetratricopeptide repeat protein 13 | sp\|Q8NBP0-2\|TTC13_HUMAN | 0.1100 |
| Transmembrane protein 106B | sp\|Q9NUM4\|T106B_HUMAN | 0.1100 |
| Neural cell adhesion molecule L1 | sp\|P32004-2\|L1CAM_HUMAN | 0.0990 |
| Scavenger receptor class A member 5 | sp\|Q6ZMJ2\|SCAR5_HUMAN | 0.0990 |
| Disintegrin and metalloproteinase domain-containing protein 9 | sp\|Q13443\|ADAM9_HUMAN | 0.0910 |
| 4F2 cell-surface antigen heavy chain | sp\|P08195-2\|4F2_HUMAN | 0.0910 |
| Metal transporter CNNM4 | sp\|Q6P4Q7\|CNNM4_HUMAN | 0.0830 |
| Secretory carrier-associated membrane protein 3 | sp\|O14828\|SCAM3_HUMAN | 0.0830 |
| Renin receptor | sp\|O75787-2\|RENR_HUMAN | 0.0660 |
| Niemann-Pick C1 protein | sp\|O15118\|NPC1_HUMAN | 0.0660 |
| Synaptonemal complex protein SC65 | sp\|Q92791\|SC65_HUMAN | 0.0660 |
| Nidogen-2 | sp\|Q14112-2\|NID2_HUMAN | 0.0660 |
| Urokinase plasminogen activator surface receptor | sp\|Q03405-2\|UPAR_HUMAN | 0.0660 |
| Epidermal growth factor receptor | sp\|P00533\|EGFR_HUMAN | 0.0580 |
| Sortilin-related receptor | sp\|Q92673\|SORL_HUMAN | 0.0580 |
| Serpin H1 | sp\|P50454\|SERPH_HUMAN | 0.0580 |
| Attractin | sp\|O75882-2\|ATRN_HUMAN | 0.0580 |
| Protocadherin Fat 1 | sp\|Q14517\|FAT1_HUMAN | 0.0580 |
| Neutral amino acid transporter B (0) | sp\|Q15758\|AAAT_HUMAN | 0.0500 |
| Integrin alpha-5 | sp\|P08648\|ITA5_HUMAN | 0.0410 |
| Cell cycle control protein 50A | sp\|Q9NV96\|CC50A_HUMAN | 0.0330 |
| Solute carrier family 2, facilitated glucose transporter member 1 | sp\|P11166\|GTR1_HUMAN | 0.0330 |
| Endothelial protein C receptor | sp\|Q9UNN8\|EPCR_HUMAN | 0.0330 |
| Insulin-like growth factor 1 receptor | sp\|P08069\|IGF1R_HUMAN | 0.0330 |
| Integrin alpha-3 | sp\|P26006\|ITA3_HUMAN | 0.0250 |
| Cation-dependent mannose-6-phosphate receptor | sp\|P20645\|MPRD_HUMAN | 0.0250 |
| Prolow-density lipoprotein receptor-related protein 1 | sp\|Q07954\|LRP1_HUMAN | 0.0170 |
| Magnesium transporter protein 1 | sp\|Q9H0U3\|MAGT1_HUMAN | 0.0170 |
| Solute carrier family 12 member 7 | sp\|Q9Y666\|S12A7_HUMAN | 0.0083 |
| Integrin alpha-2 | sp\|P17301\|ITA2_HUMAN | 0.0083 |

Co-immunoprecipitation with AvFc covalently bound to agarose beads was used to capture potential binding partners in whole-cell lysates. These proteins were then identified using mass spectrometry. The top 100 hits were then narrowed down using Gene Ontology terms as well as literature searches so as to include only transmembrane receptors, transporters, and/or adhesion molecules which may be present on the cell surface and thus which may have increased incidence of high-mannose glycans. Putative binding partners are ranked by their relative abundance within each individual analysis.

### REFERENCES

1. Loke, I, Kolarich, D, Packer, NH, and Thaysen-Andersen, M (2016). Emerging roles of protein mannosylation in inflammation and infection. Mol Aspects Med 51: 31-55.
2. Oliveira-Ferrer, L, Legler, K, and Milde-Langosch, K (2017). Role of protein glycosylation in cancer metastasis. Semin Cancer Biol.
3. Oliveira-Ferrer, L, Legler, K, and Milde-Langosch, K (2017). Role of protein glycosylation in cancer metastasis. Semin Cancer Biol 44: 141-152.
4. Pearce, OMT (2018). Cancer glycan epitopes: biosynthesis, structure and function. Glycobiology 28: 670-696.
5. de Leoz, ML, Young, LJ, An, HJ, Kronewitter, SR, Kim, J, Miyamoto, S, et al. (2010). High-mannose glycans are elevated during breast cancer progression. Mol Cell Proteomics.
6. Liu, X, Nie, H, Zhang, Y, Yao, Y, Maitikabili, A, Qu, Y, et al. (2013). Cell surface-specific N-glycan profiling in breast cancer. PLoS One 8: e72704.
7. Sethi, MK, Hancock, W S, and Fanayan, S (2016). Identifying N-Glycan Biomarkers in Colorectal Cancer by Mass Spectrometry. Acc Chem Res 49: 2099-2106.
8. Chik, JH, Zhou, J, Moh, E S, Christopherson, R, Clarke, SJ, Molloy, M P, et al. (2014). Comprehensive glycomics comparison between colon cancer cell cultures and tumours: implications for biomarker studies. J Proteomics 108: 146-162.
9. Boyaval, F, Van Zeijl, R, Dalebout, H, Holst, S, van Pelt, G, Farina-Sarasqueta, A, et al. (2021). N-glycomic signature of stage II colorectal cancer and its association with the tumor microenvironment. Mol Cell Proteomics: 100057.
10. Powers, TW, Holst, S, Wuhrer, M, Mehta, A S, and Drake, RR (2015). Two-Dimensional N-Glycan Distribution Mapping of Hepatocellular Carcinoma Tissues by MALDI-Imaging Mass Spectrometry. Biomolecules 5: 2554-2572.
11. Takayama, H, Ohta, M, Iwashita, Y, Uchida, H, Shitomi, Y, Yada, K, et al. (2020). Altered glycosylation associated with dedifferentiation of hepatocellular carcinoma: a lectin microarray-based study. BMC Cancer 20: 192.
12. Park, DD, Phoomak, C, Xu, G, Olney, LP, Tran, KA, Park, S S, et al. (2020). Metastasis of cholangiocarcinoma is promoted by extended high-mannose glycans. Proc Natl Acad Sci USA 117: 7633-7644.
13. Ruhaak, LR, Taylor, SL, Stroble, C, Nguyen, UT, Parker, EA, Song, T, et al. (2015). Differential N-Glycosylation Patterns in Lung Adenocarcinoma Tissue. J Proteome Res 14: 4538-4549.
14. Park, HM, Hwang, MP, Kim, YW, Kim, KJ, Jin, J M, Kim, Y H, et al. (2015). Mass spectrometry-based N-linked glycomic profiling as a means for tracking pancreatic cancer metastasis. Carbohydr Res 413: 5-11.
15. Everest-Dass, AV, Briggs, MT, Kaur, G, Oehler, MK, Hoffmann, P, and Packer, NH (2016). N-glycan MALDI Imaging Mass Spectrometry on Formalin-Fixed Paraffin-Embedded Tissue Enables the Delineation of Ovarian Cancer Tissues. Mol Cell Proteomics 15: 3003-3016.
16. Chen, H, Deng, Z, Huang, C, Wu, H, Zhao, X, and Li, Y (2017). Mass spectrometric profiling reveals association of N-glycan patterns with epithelial ovarian cancer progression. Tumour Biol 39: 1010428317716249.
17. Munkley, J, Mills, I G, and Elliott, DJ (2016). The role of glycans in the development and progression of prostate cancer. Nat Rev Urol 13: 324-333.
18. Moginger, U, Grunewald, S, Hennig, R, Kuo, CW, Schirmeister, F, Voth, H, et al. (2018). Alterations of the Human Skin N- and O-Glycome in Basal Cell Carcinoma and Squamous Cell Carcinoma. Front Oncol 8: 70.
19. Hamorsky, KT, Kouokam, JC, Dent, MW, Grooms, TN, Husk, A S, Hume, S D, et al. (2019). Engineering of a Lectibody Targeting High-Mannose-Type Glycans of the HIV Envelope. Mol Ther 27: 2038-2052.
20. Matoba, N, Husk, A S, Barnett, BW, Pickel, MM, Arntzen, CJ, Montefiori, D C, et al. (2010). HIV-1 neutralization profile and plant-based recombinant expression of actinohivin, an Env glycan-specific lectin devoid of T-cell mitogenic activity. PLoS One 5: e11143.
21. Seber Kasinger, LE, Dent, MW, Mahajan, G, Hamorsky, K T, and Matoba, N (2019). A novel anti-HIV-1 bispecific bNAb-lectin fusion protein engineered in a plant-based transient expression system. Plant Biotechnol J.
22. Dent, M, Hamorsky, K, Vausselin, T, Dubuisson, J, Miyata, Y, Morikawa, Y, et al. (2020). Safety and Efficacy of Avaren-Fc Lectibody Targeting HCV High-Mannose Glycans in a Human Liver Chimeric Mouse Model. Cell Mol Gastroenterol Hepatol.
23. Maley, F, Trimble, RB, Tarentino, A L, and Plummer, TH, Jr. (1989). Characterization of glycoproteins and their associated oligosaccharides through the use of endoglycosidases. Anal Biochem 180: 195-204.
24. Ludovini, V, Bellezza, G, Pistola, L, Bianconi, F, Di Carlo, L, Sidoni, A, et al. (2009). High coexpression of both insulin-like growth factor receptor-1 (IGFR-1) and epidermal growth factor receptor (EGFR) is associated with shorter disease-free survival in resected non-small-cell lung cancer patients. Ann Oncol 20: 842-849.
25. Janku, F, Garrido-Laguna, I, Petruzelka, LB, Stewart, D J, and Kurzrock, R (2011). Novel therapeutic targets in non-small cell lung cancer. J Thorac Oncol 6: 1601-1612.
26. Yeo, CD, Park, KH, Park, CK, Lee, SH, Kim, SJ, Yoon, H K, et al. (2015). Expression of insulin-like growth factor 1 receptor (IGF-1R) predicts poor responses to epidermal growth factor receptor (EGFR) tyrosine kinase inhibitors in non-small cell lung cancer patients harboring activating EGFR mutations. Lung Cancer 87: 311-317.
27. Downward, J, Parker, P, and Waterfield, MD (1984). Autophosphorylation sites on the epidermal growth factor receptor. Nature 311: 483-485.
28. Gates, R E, and King, LE, Jr. (1985). Different forms of the epidermal growth factor receptor kinase have different autophosphorylation sites. Biochemistry 24: 5209-5215.
29. Xu, Q, Malecka, KL, Fink, L, Jordan, EJ, Duffy, E, Kolander, S, et al. (2015). Identifying three-dimensional structures of autophosphorylation complexes in crystals of protein kinases. Sci Signal 8: rs13.
30. Laron, Z (2001). Insulin-like growth factor 1 (IGF-1): a growth hormone. Mol Pathol 54: 311-316.
31. Liu, Q, Guan, JZ, Sun, Y, Le, Z, Zhang, P, Yu, D, et al. (2017). Insulin-like growth factor 1 receptor-mediated cell survival in hypoxia depends on the promotion of autophagy via suppression of the PI3K/Akt/mTOR signaling pathway. Mol Med Rep 15: 2136-2142.
32. Zheng, W H, and Quirion, R (2006). Insulin-like growth factor-1 (IGF-1) induces the activation/phosphorylation of Akt kinase and cAMP response element-binding protein (CREB) by activating different signaling pathways in PC12 cells. BMC Neurosci 7: 51.
33. Yarden, Y (2001). The EGFR family and its ligands in human cancer. signalling mechanisms and therapeutic opportunities. Eur J Cancer 37 Suppl 4: S3-8.
34. Engelman, J A, and Cantley, LC (2006). The role of the ErbB family members in non-small cell lung cancers sensitive to epidermal growth factor receptor kinase inhibitors. Clin Cancer Res 12: 4372s-4376s.
35. Friedl, P, and Wolf, K (2003). Tumour-cell invasion and migration: diversity and escape mechanisms. Nat Rev Cancer 3: 362-374.
36. Gentilini, D, Busacca, M, Di Francesco, S, Vignali, M, Vigano, P, and Di Blasio, A M (2007). PI3K/Akt and ERK1/2 signalling pathways are involved in endometrial cell migration induced by 17beta-estradiol and growth factors. Mol Hum Reprod 13: 317-322.
37. Newsom-Davis, TE, Wang, D, Steinman, L, Chen, PF, Wang, LX, Simon, A K, et al. (2009). Enhanced immune recognition of cryptic glycan markers in human tumors. Cancer Res 69: 2018-2025.
38. Kiernan, JA (1975). Localization of alpha-D-glucosyl and alpha-D-mannosyl groups of mucosubstances with concanavalin A and horseradish peroxidase. Histochemistry 44: 39-45.
39. Li, J, Choi, E, Yu, H, and Bai, XC (2019). Structural basis of the activation of type 1 insulin-like growth factor receptor. Nat Commun 10: 4567.
40. Kavran, J M, McCabe, J M, Byrne, PO, Connacher, MK, Wang, Z, Ramek, A, et al. (2014). How IGF-1 activates its receptor. Elife 3.
41. Liu, P, Cleveland, TEt, Bouyain, S, Byrne, PO, Longo, P A, and Leahy, DJ (2012). A single ligand is sufficient to activate EGFR dimers. Proc Natl Acad Sci USA 109: 10861-10866.
42. Steiner, P, Joynes, C, Bassi, R, Wang, S, Tonra, JR, Hadari, Y R, et al. (2007). Tumor growth inhibition with cetuximab and chemotherapy in non-small cell lung cancer xenografts expressing wild-type and mutated epidermal growth factor receptor. Clin Cancer Res 13: 1540-1551.
43. Pujol, J L, Pirker, R, Lynch, TJ, Butts, CA, Rosell, R, Shepherd, F A, et al. (2014). Meta-analysis of individual patient data from randomized trials of chemotherapy plus cetuximab as first-line treatment for advanced non-small cell lung cancer. Lung Cancer 83: 211-218.
44. Pirker, R, Pereira, JR, von Pawel, J, Krzakowski, M, Ramlau, R, Park, K, et al. (2012). EGFR expression as a predictor of survival for first-line chemotherapy plus cetuximab in patients with advanced non-small-cell lung cancer: analysis of data from the phase 3 FLEX study. Lancet Oncol 13: 33-42.
45. Pirker, R, Pereira, JR, Szczesna, A, von Pawel, J, Krzakowski, M, Ramlau, R, et al. (2009). Cetuximab plus chemotherapy in patients with advanced non-small-cell lung cancer (FLEX): an open-label randomised phase III trial. Lancet 373: 1525-1531.
46. Morgillo, F, Kim, WY, Kim, E S, Ciardiello, F, Hong, W K, and Lee, HY (2007). Implication of the insulin-like growth factor-IR pathway in the resistance of non-small cell lung cancer cells to treatment with gefitinib. Clin Cancer Res 13: 2795-2803.
47. Kjaer, I, Lindsted, T, Frohlich, C, Olsen, JV, Horak, ID, Kragh, M, etal. (2016). Cetuximab Resistance in Squamous Carcinomas of the Upper Aerodigestive Tract Is Driven by Receptor Tyrosine Kinase Plasticity: Potential for mAb Mixtures. Mol Cancer Ther 15: 1614-1626.
48. Huang, F, Xu, LA, and Khambata-Ford, S (2012). Correlation between gene expression of IGF-1R pathway markers and cetuximab benefit in metastatic colorectal cancer. Clin Cancer Res 18: 1156-1166.
49. Guo, XF, Zhu, XF, Cao, HY, Zhong, GS, Li, L, Deng, B G, et al. (2017). A bispecific enediyne-energized fusion protein targeting both epidermal growth factor receptor and insulin-like growth factor 1 receptor showing enhanced antitumor efficacy against non-small cell lung cancer. Oncotarget 8: 27286-27299.
50. Al-Saad, S, Richardsen, E, Kilvaer, TK, Donnem, T, Andersen, S, Khanehkenari, M, et al. (2017). The impact of MET, IGF-1, IGF1R expression and EGFR mutations on survival of patients with non-small-cell lung cancer. PLoS One 12: e0181527.
51. Wong, MY, Chen, K, Antonopoulos, A, Kasper, BT, Dewal, MB, Taylor, R J, et al. (2018). XBP1s activation can globally remodel N-glycan structure distribution patterns. Proc Natl Acad Sci USA 115: E10089-E10098.
52. Zhang, G, Isaji, T, Xu, Z, Lu, X, Fukuda, T, and Gu, J (2019). N-acetylglucosaminyltransferase-I as a novel regulator of epithelial-mesenchymal transition. FASEB J 33: 2823-2835.
53. Alonso-Garcia, V, Chaboya, C, Li, Q, Le, B, Congleton, TJ, Florez, J, et al. (2020). High Mannose N-Glycans Promote Migration of Bone-Marrow-Derived Mesenchymal Stromal Cells. Int J Mol Sci 21.
54. Strasser, R. et al. (2008) Generation of glyco-engineered Nicotiana benthamiana for the production of monoclonal antibodies with a homogeneous human-like N-glycan structure. Plant Biotechnol J 6, 392-402.
55. Johns, T. G. et al. (2005) The antitumor monoclonal antibody 806 recognizes a high-mannose form of the EGF receptor that reaches the cell surface when cells over-express the receptor. FASEB J 19, 780-782.
56. Sato, Y. et al. (2016) High mannose-binding Pseudomonas fluorescens lectin (PFL) downregulates cell surface integrin/EGFR and induces autophagy in gastric cancer cells. BMC Cancer 16, 63.
57. Hasegawa, Y. et al. (2015) Surfactant protein D suppresses lung cancer progression by downregulation of epidermal growth factor signaling. Oncogene 34, 4285-4286.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of reducing activation of a cancer-associated growth factor receptor in a cancer cell, comprising contacting the cancer cell with a polypeptide that specifically binds a high-mannose-type glycan epitope.

2. The method of claim 1, wherein the polypeptide is in an amount sufficient to reduce activation of the cancer-associated growth factor receptor in the cancer cell by about 10-90%.

3. The method of claim 1, wherein the cancer-associated growth factor receptor comprises at least one tumor-associated growth factor receptor.

4. The method of claim 1, wherein the cancer-associated growth factor receptor comprises an epidermal growth factor receptor (EGFR), an insulin-like growth factor 1 receptor (IGF1R), or a combination thereof.

5. The method of claim 1, wherein the cancer cell is an in vitro cell or an ex vivo cell.

6. (canceled)

7. The method of claim 1, wherein the cancer cell is a lung cancer cell.

8. The method of claim 1, wherein the cancer cell is a non-small cell lung cancer (NSCLC) cell.

9. A method of treating cancer in a subject in need thereof, comprising:
a) providing a biological sample from the subject;
b) determining presence or absence of an abnormal accumulation of a high-mannose glycan epitope in the biological sample; and
c) administering or providing for administration a therapeutically effective amount of a polypeptide that specifically binds the high-mannose-type glycan epitope to the subject if the abnormal accumulation is determined to be present in the biological sample.

10. A method of treating cancer in a subject in need thereof, comprising administering to the subject an effective amount of a polypeptide that specifically binds a high-mannose-type glycan epitope, wherein the cancer is characterized by an abnormal cell-surface accumulation of high-mannose glycans, or wherein the cancer is mediated by inappropriate activation of a growth factor receptor.

11. (canceled)

12. The method of claim 10, wherein the growth factor receptor comprises an epidermal growth factor receptor (EGFR), an insulin-like growth factor 1 receptor (IGF1R), or a combination thereof.

13. The method of claim 9, wherein the cancer is a lung cancer.

14. The method of claim 13, wherein the lung cancer is non-small cell lung cancer (NSCLC).

15. The method of claim 9, wherein the cancer is resistant to treatment with an antibody that specifically binds a growth factor receptor.

16. The method of claim 15, wherein the subject is resistant to treatment with an antibody that specifically binds an epidermal growth factor receptor (EGFR).

17. The method of claim 9, wherein the therapeutically effective amount of the polypeptide is sufficient to:
a) reduce activation of a growth factor receptor;
b) inhibit cancer cell migration;
c) induce a cytotoxic effect; or
d) slow tumor growth,
in the subject, or a combination thereof.

18. The method of claim 1, wherein the polypeptide comprises an amino acid sequence that is at least about 90% identical to at least one sequence set forth in SEQ ID NOs:1-13.

19. The method of claim 18, wherein the polypeptide comprises an amino acid sequence set forth in SEQ ID NO:9.

20. The method of claim 1, wherein the polypeptide further comprises a fragment crystallizable domain of an antibody (Fc), a fragment antigen-binding domain of an antibody (Fab) or a single chain variable fragment of an antibody (scFv).

21. The method of claim 20, wherein the polypeptide further comprises an Fc.

22. The method of claim 1, wherein the polypeptide comprises an amino acid sequence set forth in SEQ ID NO:16.
